(19) United States
(12) Patent Application Publication
(10) Pub. No.: US 2024/0161290 A1
(43) Pub. Date: May 16, 2024

(54) GENERATING COUNTERFACTUAL EXPLANATIONS OF TUMOR SPATIAL PROTEOMES TO ENHANCE CANCER IMMUNOTHERAPY

(71) Applicant: California Institute of Technology, Pasadena, CA (US)

(72) Inventors: Matthew W. Thomson, Pasadena, CA (US); Zitong Wang, Pasadena, CA (US)

(21) Appl. No.: 18/504,053

(22) Filed: Nov. 7, 2023

Related U.S. Application Data
(60) Provisional application No. 63/423,373, filed on Nov. 7, 2022.

(51) Int. Cl. G06T 7/00; G06V 10/762; G06V 10/764; G06V 10/774; G06V 10/82; G06V 20/69; G06V 20/70; G16H 30/40; G16H 50/20

(52) U.S. Cl. CPC G06T 7/0012; G06V 10/762; G06V 10/764; G06V 10/774; G06V 10/82; G06V 20/698; G06V 20/70; G16H 30/40; G16H 50/20; G06T 2207/20081; G06T 2207/20084; G06T 2207/30096

(57) ABSTRACT

Disclosed herein include systems, devices, and methods for counterfactual optimization. In some embodiments, spatial omics training data can comprise a plurality of training images each comprising a plurality of molecule channels. A training image label can be generated for each of a plurality of training images indicating presence of at least one T cell in the training image. A plurality of masked training images can be generated from a plurality of training images with any T cell present in a training image of the plurality of training images masked in a masked training image of the plurality of masked training images generated. A model comprising a classifier with the plurality of masked training images as input and the training image label as output can be generated. A counterfactual optimization can be performed to determine a tumor perturbation using a second plurality of images with no T cell present in each of the plurality of images.

### RELATED APPLICATIONS

This application claims the benefit under 35 U.S.C. § 119(e) of U.S. Provisional Patent Application Ser. No. 63/423,373, filed Nov. 7, 2022, the content of this related application is incorporated herein by reference in its entirety for all purposes.

### BACKGROUND

### Field

The present disclosure relates generally to the field of enhancing cancer immunotherapy.

### Description of the Related Art

The immune composition of the tumor microenvironment (TME) plays a crucial role in determining patient prognosis and responses to cancer immunotherapies. For immunotherapies to be effective, T cells must be able to infiltrate the tumor microenvironment (TME). However, many solid tumors resist T-cell infiltration, challenging the efficacy of current therapies. Immune cell localization such as CD8+ T cells can be important in determining patient outcomes. There is a need to determine immune cell localization from environmental signals.

### SUMMARY

Disclosed herein include systems for counterfactual optimization. In some embodiments, a system for counterfactual optimization comprises: non-transitory memory configured to store: executable instructions. The non-transitory memory can be configured to store: spatial omics training data comprising a plurality of training images each comprising a plurality of molecule channels. The system can comprise: a processor (e.g., a hardware processor or a virtual processor) in communication with the non-transitory memory, the processor programmed by the executable instructions to perform: generating a training image label for each of the plurality of training images indicating presence of at least one T cell in the training image. The processor can be programmed by the executable instructions to perform: generating a plurality of masked training images from the plurality of training images with any T cell present in a training image of the plurality of training images masked in a masked training image of the plurality of masked training images generated. The processor can be programmed by the executable instructions to perform: training a model comprising a classifier with the plurality of masked training images as input and the training image label as output. The processor can be programmed by the executable instructions to perform: performing a counterfactual optimization to determine a tumor perturbation using a second plurality of images with no T cell present in each of the plurality of images. In some embodiments, the processor is programmed by the executable instructions to perform: receiving the second plurality of images with no T cell present in each of the plurality of images. In some embodiments, the non-transitory memory is configured to store: the second plurality of images with no T cell present in each of the plurality of images.

In some embodiments, the T cell is CD8+ T cell. In some embodiments, the spatial omics training data comprises spatial omics data generated from a tumor sample of a subject. The spatial omics training data can comprise spatial omics data generated from a tumor sample for each of a plurality of subjects. In some embodiments, the spatial omics training data comprises spatial omics data generated from a plurality of tumor samples of a subject. The spatial omics training data can comprise spatial omics data generated from a plurality of tumor samples for each of a plurality of subjects. In some embodiments, a subject comprises a mammal, or a human. In some embodiments, the subject is a cancer subject. In some embodiments, the number of the plurality of subjects can be, be about, be at least, be at least about, be at most, or be at most about, 25, 50, 75, 100, 200, 300, 400, 500, 600, 700, 800, 900, 1000, 2500, 5000, 7500, 10000, 25000, 50000, 75000, 100000, or a number of a range between any two of these values. In some embodiments, the number of tumor samples of a subject can be, be about, be at least, be at least about, be at most, or be at most about, 1, 2, 3, 4, 5, 6, 7, 8, 9, 10, or a number of a range between any two of these values. In some embodiments, the number of the plurality of training images can be, be about, be at least, be at least about, be at most, or be at most about, 25, 50, 75, 100, 200, 300, 400, 500, 600, 700, 800, 900, 1000, 2500, 5000, 7500, 10000, 25000, 50000, 75000, 100000, 250000, 500000, 750000, 1000000 or a number of a range between any two of these values. In some embodiments, the number of training images of a subject (or per subject on average) can be, be about, be at least, be at least about, be at most, or be at most about, 25, 50, 75, 100, 200, 300, 400, 500, 600, 700, 800, 900, 1000, 2500, 5000, 7500, 10000, 25000, 50000, 75000, 100000, or a number of a range between any two of these values.

In some embodiments, the spatial omics data is generated using imaging mass cytometry. In some embodiments, the spatial omics data comprises proteinomics data, transcriptomics data, or a combination thereof. In some embodiments, the spatial omics data comprises multi-omics training data.

In some embodiments, a training image of the plurality of training images is, is about, is at least, is at least about, is at most, or is at most about, 20, 25, 30, 35, 40, 45, 58, 50, 55, 60, 70, 80, 90, 100, 125, 150, 175, 200, 250, 300, or a number or a range between any two of these values, pixels in length (or width). In some embodiments, a training image of the plurality of training images corresponds to a section (or a region) that is, is about, is at least, is at least about, is at most, or is at most about, 20 μm, 25 μm, 30 μm, 35 μm, 40 μm, 45 μm, 58 μm, 50 μm, 55 μm, 60 μm, 70 μm, 80 μm, 90 μm, 100 μm, 125 μm, 150 μm, 175 μm, 200 μm, 250 μm, 300 μm, or a number or a range between any two of these values, in length (or width).

In some embodiments, the plurality of molecule channels comprises, comprises about, comprises at least, comprises at least about, comprises at most, or comprises at most about, 10, 15, 20, 25, 30, 35, 37 40, 41, 45, 50, 55, 60, 65, 70, 75, 80, 85, 90, 95, 100, 125, 150, 175, 200, 250, 300, 400, 500, or a number or a range between any two of these values, channels. In some embodiments, each of the plurality of molecule channels corresponds to a different protein. In some embodiments, the molecules comprise chemokines. In some embodiments, the molecules comprise CXCL9, CXCL10, CCL18, CCL22, or a combination thereof. In some embodiments, the molecules comprise Histone H3, SMA, CK5, CD38, HLA-DR, CK8-18, CD15, FSPI, CD163, ICOS, OX40, CD68, HER2 (3B5), CD3, Podoplanin, CD11c, PD-1, GITR, CD16, HERZ (D8F12), CD45RA, B2M, CD45RO, FOXP3, CD20, ER, CD57, Ki-67, PDGFRB, Caveolin-1, CD4, CD31-VWE, CXCL12, HLA-ABC, panCK, c-Caspase3, DNA1, DNA2, CD8, or a combination thereof.

In some embodiments, the training image label is a binary value. The training image label being 0 can indicate absence of any T cell in a corresponding training image of the training image label. The training image label being 1 can indicate presence of at least one T cell in a corresponding training image of the training image label. In some embodiments, generating the training image label comprises: generating the training image label by clustering cells in the training image and one or more other (e.g., 2, 3, 4, 5, 10, 25, 50, 75, 100, or all or substantially all) training images of the plurality of training images.

In some embodiments, masked pixels in one, one or more, or each of the plurality of masked training images comprise values of 0. Masked pixels in one, one or more, or each of the plurality of masked training images can comprise an average value of pixels (e.g., some pixels, pixels excluding the masked pixels, or all pixels) in the masked training image or the corresponding training image. In some embodiments, the plurality of masked training images comprises, for each of the plurality training images, the training image if no T cell is present in the training image, or the training image with any T cell present in the training image masked.

In some embodiments, the model comprises a fully-connected layer connected to a last layer of the classifier. The fully-connected layer can output a value between 0 and 1. The model can output a value of 0 if the output of the fully-connected layer is below a threshold value. The model can output a value of 1 if the output of the fully-connected layer is at least the threshold value. In some embodiments, wherein the threshold value is 0.9 (or is, is about, is at least, is at least about, is at most, or is at most about, 7, 7.5, 8, 8.5, 8.6, 8.7, 8.8, 8.9, 9, 9.1, 9.2, 9.3, 9.4, 9.5, or a number or a range between any two of these values). In some embodiments, the processor is programmed by the executable instructions to perform: determining the threshold value, optionally wherein determining the threshold value using root mean squared error (RMSE).

In some embodiments, the classifier comprises a neural network, a deep neural network, a convolutional neural network, a fully convolutional neural network, or a combination thereof. In some embodiments, the classifier comprises U-Net, Reset-18, EfficientNet-B0, MedViT, or a combination thereof. In some embodiments, training the model comprises training the model using stochastic gradient decent and/or T cell prediction loss. In some embodiments, the spatial omics training data comprises the second plurality of images with no T cell present in each the image. In some embodiments, the plurality of training images comprises the second plurality of images with no T cell present in the image. In some embodiments, the second plurality of images with no T cell present in the image comprises no training image of the plurality of training images.

In some embodiments, performing the counterfactual optimization comprises: performing the counterfactual optimization using a term corresponding to increasing predicted probability of T cells, a term corresponding to minimizing change, and/or a term corresponding to a shift closer to training data. In some embodiments, a tumor perturbation comprises, for each of the second plurality of images, a change in an intensity in one or more (e.g., 1, 2, 3, 4, 5, 6, 7, 8, 9, 10, 15, 20, 25, 30, 35, 40, 45, 50, 60, 70, 80, 90, or 100, or all, or a number or a range between any two of these values) of the plurality of molecule channels. In some embodiments, the processor is programmed by the executable instructions to perform: applying the tumor perturbation to a plurality of test images with a T-cell distribution to determine a perturbed T-cell distribution. The tumor perturbation can be tested, for example, on cells, an organoid, a tissue, or a combination thereof.

Disclosed herein include methods for counterfactual optimization. In some embodiments, a method for counterfactual optimization is under control of a processor (e.g., a hardware processor or a virtual processor) and comprises: receiving spatial omics training data comprising a plurality of training images each comprising a plurality of molecule channels. The method can comprise: generating a training image label for each of the plurality of training images indicating presence of at least one T cell in the training image. The method can comprise: generating a plurality of masked training images from the plurality of training images with any T cell present in a training image of the plurality of training images masked in a masked training image of the plurality of masked training images generated. The method can comprise: training a model comprising a classifier with the plurality of masked training images as input and the training image label as output. The method can comprise: performing a counterfactual optimization to determine a tumor perturbation using a second plurality of images with no T cell present in each of the plurality of images. In some embodiments, the method comprises: receiving the second plurality of images with no T cell present in each of the plurality of images

In some embodiments, the spatial omics data is generated using imaging mass cytometry. In some embodiments, the spatial omics data comprises proteinomics data, transcriptomics data, or a combination thereof. In some embodiments, the spatial omics data comprises multi-omics training data.

In some embodiments, a training image of the plurality of training images is, is about, is at least, is at least about, is at most, or is at most about, 20, 25, 30, 35, 40, 45, 58, 50, 55, 60, 70, 80, 90, 100, 125, 150, 175, 200, 250, 300, or a number or a range between any two of these values, pixels in length (or width). In some embodiments, a training image of the plurality of training images corresponds to a section (or a region) that is, is about, is at least, is at least about, is at most, or is at most about, 20 μm, 25 μm, 30 μm, 35 μm, 40 μm, 45 μm, 58 μm, 50 μm, 55 μm, 60 μm, 70 μm, 80 μm, 90 μm, 100 μm, 125 μm, 150 μm, 175 μm, 200 μm, 250 μm, 300 μm, or a number or a range between any two of these values, in length (or width).

In some embodiments, the plurality of molecule channels comprises, comprises about, comprises at least, comprises at least about, comprises at most, or comprises at most about, 10, 15, 20, 25, 30, 35, 37 40, 41, 45, 50, 55, 60, 65, 70, 75, 80, 85, 90, 95, 100, 125, 150, 175, 200, 250, 300, 400, 500, or a number or a range between any two of these values, channels. In some embodiments, each of the plurality of molecule channels corresponds to a different protein. In some embodiments, the molecules comprise chemokines. In some embodiments, the molecules comprise CXCL9, CXCL10, CCL18, CCL22, or a combination thereof. In some embodiments, the molecules comprise Histone H3, SMA, CK5, CD38, HLA-DR, CK8-18, CD15, FSPI, CD163, ICOS, OX40, CD68, HER2 (3B5), CD3, Podoplanin, CD11c, PD-1, GITR, CD16, HERZ (D8F12), CD45RA, B2M, CD45RO, FOXP3, CD20, ER, CD57, Ki-67, PDGFRB, Caveolin-1, CD4, CD31-VWE, CXCL12, HLA-ABC, panCK, c-Caspase3, DNA1, DNA2, CD8, or a combination thereof.

In some embodiments, the training image label is a binary value. The training image label being 0 can indicate absence of any T cell in a corresponding training image of the training image label. The training image label being 1 can indicate presence of at least one T cell in a corresponding training image of the training image label. In some embodiments, generating the training image label comprises: generating the training image label by clustering cells in the training image and one or more other (e.g., 2, 3, 4, 5, 10, 25, 50, 75, 100, or all or substantially all) training images of the plurality of training images.

In some embodiments, masked pixels in one, one or more, or each of the plurality of masked training images comprise values of 0. Masked pixels in one, one or more, or each of the plurality of masked training images can comprise an average value of pixels (e.g., some pixels, pixels excluding the masked pixels, or all pixels) in the masked training image or the corresponding training image. In some embodiments, the plurality of masked training images comprises, for each of the plurality training images, the training image if no T cell is present in the training image, or the training image with any T cell present in the training image masked.

In some embodiments, the model comprises a fully-connected layer connected to a last layer of the classifier. The fully-connected layer can output a value between 0 and 1. The model can output a value of 0 if the output of the fully-connected layer is below a threshold value. The model can output a value of 1 if the output of the fully-connected layer is at least the threshold value. In some embodiments, wherein the threshold value is 0.9 (or is, is about, is at least, is at least about, is at most, or is at most about, 7, 7.5, 8, 8.5, 8.6, 8.7, 8.8, 8.9, 9, 9.1, 9.2, 9.3, 9.4, 9.5, or a number or a range between any two of these values). In some embodiments, the method comprises: determining the threshold value, optionally wherein determining the threshold value using root mean squared error (RMSE).

In some embodiments, the classifier comprises a neural network, a deep neural network, a convolutional neural network, a fully convolutional neural network, or a combination thereof. In some embodiments, the classifier comprises U-Net, Reset-18, EfficientNet-B0, MedViT, or a combination thereof. In some embodiments, training the model comprises training the model using stochastic gradient decent and/or T cell prediction loss. In some embodiments, the spatial omics training data comprises the second plurality of images with no T cell present in each the image. In some embodiments, the plurality of training images comprises the second plurality of images with no T cell present in the image. In some embodiments, the second plurality of images with no T cell present in the image comprises no training image of the plurality of training images.

In some embodiments, performing the counterfactual optimization comprises: performing the counterfactual optimization using a term corresponding to increasing predicted probability of T cells, a term corresponding to minimizing change, and/or a term corresponding to a shift closer to training data. In some embodiments, a tumor perturbation comprises, for each of the second plurality of images, a change in an intensity in one or more (e.g., 1, 2, 3, 4, 5, 6, 7, 8, 9, 10, 15, 20, 25, 30, 35, 40, 45, 50, 60, 70, 80, 90, or 100, or all, or a number or a range between any two of these values) of the plurality of molecule channels. In some embodiments, method comprises: applying the tumor perturbation to a plurality of test images with a T-cell distribution to determine a perturbed T-cell distribution. In some embodiments, the method comprises: testing the tumor perturbation, for example, on cells, an organoid, a tissue, or a combination thereof.

Disclosed herein include embodiments of a computer readable medium. In some embodiments, a computer readable medium comprising executable instructions, when executed by a processor (e.g., a hardware processor or a virtual processor or a virtual processor) of a computing system or a device, cause the processor, to perform any method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1A-FIG. 1C show non-limiting exemplary workflows of self-supervised training of T cell localization classifier and counterfactual optimization of tissue perturbation.

FIG. 2A-FIG. 2D illustrate non-limiting exemplary performance of U-Net classifier trained on IMC images of melanoma, metastatic liver tumor, and breast tumor that accurately predicted T cell distribution.

FIG. 3A-FIG. 3I illustrate non-limiting exemplary combinatorial chemokine therapy predicted to drive T cell infiltration in groups of metastatic melanoma patients.

FIG. 4A-FIG. 4H show non-limiting exemplary blocking subsets of PD-L1, CXCR4, PD-1, and CYR61 predicted to drive T cell infiltration in CRC cohort.

FIG. 5 illustrates non-limiting exemplary correlation between each frequency band of each protein channel and T-cell infiltration level (proportion of CD8+ T-cell patches) across all IMC images for the breast cancer data set.

FIG. 6 is a block diagram of an illustrative computing system that can be used in some embodiments to execute the processes and implement the features described herein, for example, counterfactual optimization.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Immunotherapies reverse cancer progression by activating either endogenous or engineered T cells to detect and kill tumors. For immunotherapies to be effective, T cells must be able to infiltrate the tumor microenvironment (TME). However, many solid tumors resist T-cell infiltration, challenging the efficacy of current therapies. Disclosed herein include systems, devices, and methods to formulate T-cell infiltration prediction, referred to herein as Morpheus. A deep learning framework that leverages large scale spatial omics profiles of patient tumors can be used to formulate T-cell infiltration prediction. The deep learning framework can utilize a self-supervised machine learning method. The deep learning framework in combination with a counterfactual optimization strategy ca enable the design of tumor perturbations predicted to boost T-cell infiltration. In some embodiments, combinatorial perturbations can be predicted to support T-cell infiltration across patients, such as tens to hundreds of patients. The paradigm presented here includes counterfactual-based prediction and design of cancer therapeutics using spatial omics data.

Disclosed herein include systems for counterfactual optimization. In some embodiments, a system for counterfactual optimization comprises: non-transitory memory configured to store: executable instructions. The non-transitory memory can be configured to store: spatial omics training data comprising a plurality of training images each comprising a plurality of molecule channels. The system can comprise: a processor (e.g., a hardware processor or a virtual processor) in communication with the non-transitory memory, the processor programmed by the executable instructions to perform: generating a training image label for each of the plurality of training images indicating presence of at least one T cell in the training image. The processor can be programmed by the executable instructions to perform: generating a plurality of masked training images from the plurality of training images with any T cell present in a training image of the plurality of training images masked in a masked training image of the plurality of masked training images generated. The processor can be programmed by the executable instructions to perform: training a model comprising a classifier with the plurality of masked training images as input and the training image label as output. The processor can be programmed by the executable instructions to perform: performing a counterfactual optimization to determine a tumor perturbation using a second plurality of images with no T cell present in each of the plurality of images.

Disclosed herein include methods for counterfactual optimization. In some embodiments, a method for counterfactual optimization is under control of a processor (e.g., a hardware processor or a virtual processor) and comprises: receiving spatial omics training data comprising a plurality of training images each comprising a plurality of molecule channels. The method can comprise: generating a training image label for each of the plurality of training images indicating presence of at least one T cell in the training image. The method can comprise: generating a plurality of masked training images from the plurality of training images with any T cell present in a training image of the plurality of training images masked in a masked training image of the plurality of masked training images generated. The method can comprise: training a model comprising a classifier with the plurality of masked training images as input and the training image label as output. The method can comprise: performing a counterfactual optimization to determine a tumor perturbation using a second plurality of images with no T cell present in each of the plurality of images.

### Overview

The immune composition of the tumor microenvironment (TME) plays a crucial role in determining patient prognosis and responses to cancer immunotherapies. Immunotherapies reverse cancer progression by activating either endogenous or engineered T cells to detect and kill tumors. Immunotherapies that alter the immune composition using transplanted or engineered immune cells (e.g., chimeric antigen receptor T cell therapy) or remove immunosuppressive signaling (e.g., checkpoint inhibitors) have shown exciting results in relapsed and refractory tumors in hematological cancers and some solid tumors. For immunotherapies to be effective, T cells must be able to infiltrate the tumor microenvironment (TME). However, many solid tumors resist T-cell infiltration, challenging the efficacy of current therapies. Therefore, effective therapeutic strategies for most solid tumors remain limited. The TME is a complex mixture of immune cells, including T cells, B cells, natural killer cells, and macrophages, as well as stromal cells and tumor cells. The interactions between these cells can either promote or suppress tumor growth and progression, and ultimately impact treatment outcomes in patients. For example, high levels of tumor-infiltrating lymphocytes (TILs) in the TME are associated with improved prognosis and response to immunotherapy across multiple cancer types. Conversely, an immunosuppressive TME characterized by low levels of TILs is associated with poor prognosis and reduced response to immunotherapy. Durable, long-term clinical response to T-cell-based immunotherapies are often constrained by a lack of T-cell infiltration into the tumor, as seen in classically “cold” tumors such as triple-negative breast cancer (TNBC) or pancreatic cancer, which have seen little benefit from immunotherapy.

Spatial omics technologies capture the spatial organization of cellular activity in intact human tumors with unprecedented molecular detail, revealing the relationship between localization of different cell types and tens to thousands of molecular signals, paving the way for the design of novel strategies to manipulate the tumor immune microenvironment. T-cell infiltration is modulated by a rich array of signals within the TME such as chemokines, adhesion molecules, tumor antigens, immune checkpoints, and their cognate receptors. Recent advances in in situ molecular profiling techniques, including spatial transcriptomic and proteomic methods, can simultaneously capture the spatial relationship of tens to thousands of molecular signals and T cell localization in intact human tumors with micron-scale resolution. Imaging mass cytometry (IMC) is one such technology that uses metal-labeled antibodies to enable simultaneous detection of up to 40 antigens and transcripts in intact tissue.

Recent work on computational methods, as applied to multiplexed tumor images, have primarily focused on predicting patient-level phenotypes such as survival, by identifying spatial motifs from tumor microenvironments. These methods have generated valuable insights into how the complex composition of TMEs influences patient prognosis and treatment response, but fall short of generating concrete, testable hypothesis for therapeutic interventions that can improve patient outcome. Given the importance of immune cell localization such as CD8+ T cells in determining patient outcomes, computational tools are needed, which can predict immune cell localization from environmental signals and systematically generate specific, feasible tumor perturbations predicted to alter the TME so as to improve patient outcome.

Disclosed herein is Morpheus, a deep learning framework that can leverage large scale spatial omics profiles of patient tumors to formulate T-cell infiltration prediction as a self-supervised machine learning (ML) problem, and combine this prediction task with a counterfactual optimization strategy to enable the design of tumor perturbations predicted to boost T-cell infiltration. Specifically, a convolutional neural network was trained to predict T-cell infiltration based upon micron-resolution maps of the signaling molecules in the TME provided by IMC. Then, a gradient-based counterfactual generation strategy was applied to the infiltration neural network to compute perturbations to the signaling molecules that the network predicted would increase T-cell abundance. This framework was applied to 1,117 melanoma, colorectal (with liver metastases) and breast human cancer samples assayed using IMC. Specifically, Morpheus was applies to spatial proteomic profiles of tumors from melanoma and colorectal cancers (CRC) with liver metastases, to discover tumor perturbations that were predicted to support T cell infiltration in tens to hundreds of patients across two distinct cancer types. Further validation of ML-based T-cell infiltration prediction was provided using an additional breast cancer data set. In melanoma patients, Morpheus predicted combinatorial perturbation to the CXCL9, CXCL10, CCL22 and CCL18 levels in tumor can convert immune-excluded tumors to immune-inflamed in a cohort of 69 patients. For CRC with liver metastasis, Morpheus discovered two cohort-dependent therapeutic strategies consisting of blocking different subsets of CXCR4, PD1, PDL1 and CYR61 that were predicted to consistently improve T-cell infiltration across a cohort of 30 patients. Thus, this framework provided a paradigm for counterfactual-based prediction and design of cancer therapeutics based on classification of immune system activity in spatial proteomics and transcriptomic data.

### Generating Counterfactual Explanations of Tumor Spatial Proteomes to Discover Effective Strategies for Enhancing Immune Infiltration

### A Counterfactual Optimization Framework for In Situ Molecular Profiles Enables the Discovery of Tumor Perturbations Predicted to Drive T Cell Infiltration

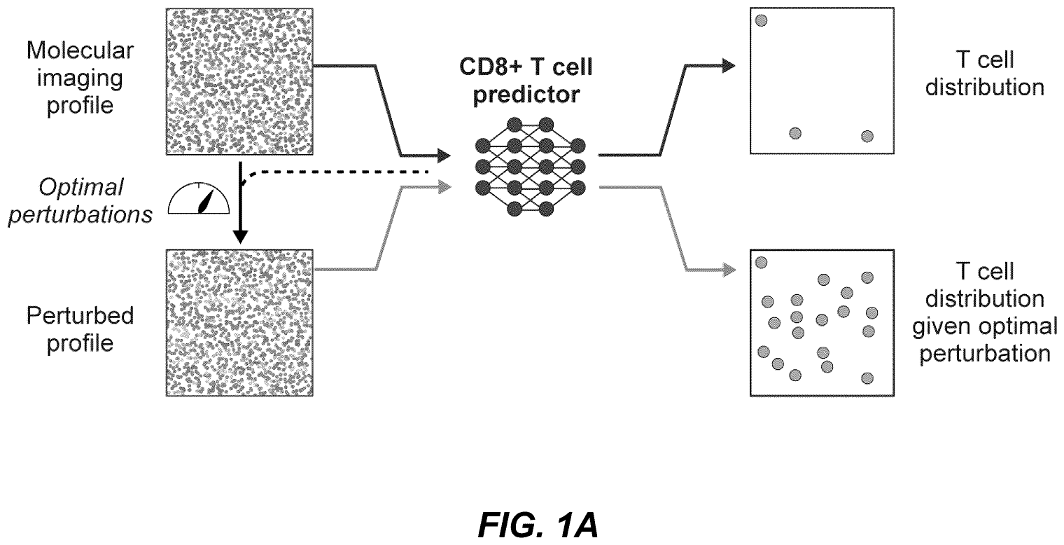

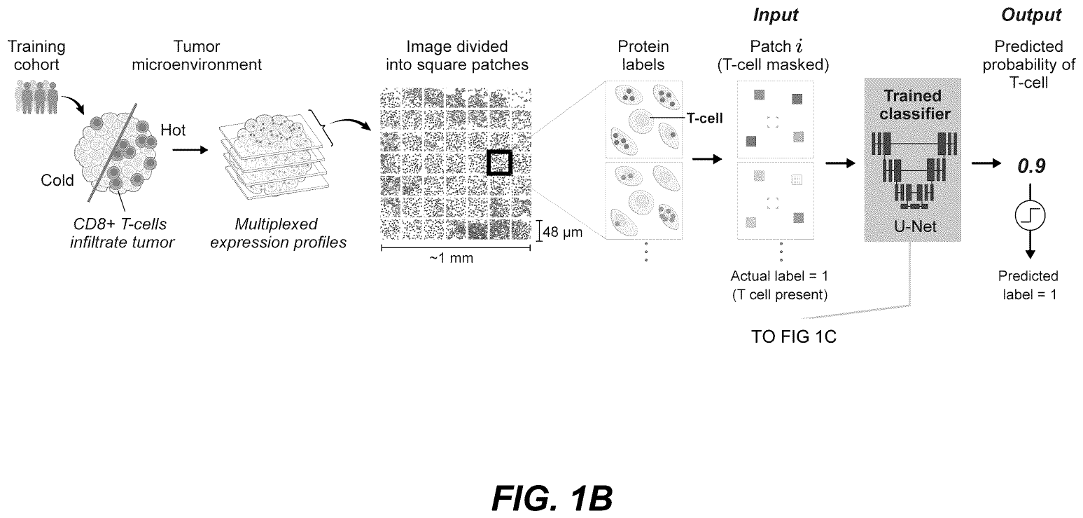

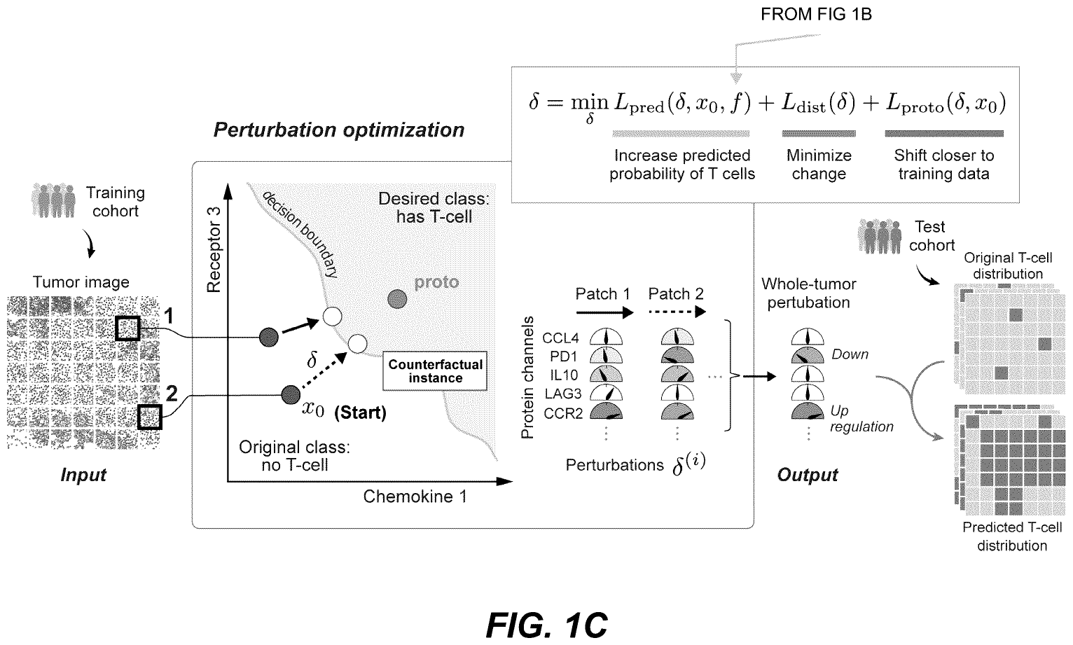

The general logic of Morpheus was to train, in a self-supervised manner, a classifier to predict the presence of CD8+ T cells from tissue images (FIG. 1A and FIG. 1B). Then, counterfactual instances of the data were discovered by performing gradient descent on the input image, allowing the discovery of perturbations to the tumor image that increased the classifier's predicted likelihood of CD8+ T cells being present (FIG. 1A and FIG. 1C). The altered image represented a perturbation of the TME predicted to improve T cell infiltration. CD8+ T cells were masked from all images to prevent the classifier from simply memorizing the T cells' expression patterns, guiding it instead to learn environmental features indicative of T cell presence.

IMC profiles of human tumors were leveraged to train a classifier to predict the spatial distribution of CD8+ T cell in a self-supervised manner. A set of images {I⁽ⁱ⁾} was considered, which was obtained by dividing IMC profiles of tumor sections into local patches of tissue signaling environments, where I⁽ⁱ⁾∈ [symbol] ^{l×w×c} is an array with l and w denoting the pixel length and width of the image and c denoting the number of molecule channels in the images (FIG. 1B). Each image showed the level of c proteins across all cells within a small patch of tissue. From patch I⁽ⁱ⁾, a binary label s⁽ⁱ⁾ indicating the presence and absence of CD8+ T cells in the patch and a masked copy x⁽ⁱ⁾ with all signals originating from CD8+ T cells removed were obtained. The task for the model f was to classify whether T cells were present (s⁽ⁱ⁾=1) or absent (s⁽ⁱ⁾=0) in image I⁽ⁱ⁾ using only its masked copy x⁽ⁱ⁾. Specifically, f(x⁽ⁱ⁾)∈[0,1] was the predicted probability of T cells. Then, a classification threshold p was applied to convert this probability to a predicted label ŝ⁽ⁱ⁾∈{0,1}. Since the image label s⁽ⁱ⁾ was obtained from the image I⁽ⁱ⁾ itself by unsupervised clustering of individual cells, the overall task was inherently self-supervised.

Given a set of image patches, a model f was trained to minimize the following T cell prediction loss, also known as the binary cross entropy (BCE) loss,

$$L = - \frac{1}{N} {\sum }_{i = 1}^{N} [ {s}^{( i )} log ( {\hat{s}}^{( i )} ) + ( 1 - {s}^{( i )} ) log ( 1 - {\hat{s}}^{( i )} ) ] , \qquad ( 1 )$$

$$where$$

$$\hat{s} ( i ) = \{ \begin{matrix}1 & if f ( {x}^{( i )} ) \geq p \\ 0 & if f ( {x}^{( i )} ) < p\end{matrix} \qquad ( 2 )$$

and p is the classification threshold. p was selected by minimizing the following root mean squared error (RMSE) on a separate set of tissue sections Ω.

$$R M S {E}^{2} = \frac{1}{\vert Ω \vert} {\sum }_{j ∈ Ω} {‖ \frac{1}{{N}_{j}} {\sum }_{i = 1}^{{N}_{j}} {\hat{s}}^{( i )} - {s}^{( i )} ‖}^{2} . \qquad ( 3 )$$

The RMSE was a measure of the discrepancies between the observed and predicted proportions of T cell patches in a tissue section averaged across a set of tissues, which was taken as the validation set.

The performance of various classifiers was evaluated, including both traditional convolutional neural networks (CNNs) and vision transformers. In all cases, similar performance was observed (Table 6). A U-Net architecture was settled on because of ease of extension of the model to multichannel data sets. The U-Net classifier consisted of a standard U-Net architecture and a fully-connected layer with softmax activation. To increase the number of samples available for training, the spatial heterogeneity of TMEs was taken advantage of and each tissue image was divided into 48 μm×48 μm (or 50×50) patches upon which the classifier was trained to predict T cell presence.

Using the trained classifier and IMC images of tumors, a counterfactual optimization method was employed to predict tumor perturbations that enhanced CD8+ T cell infiltration (FIG. 1C). For each image patch x₀⁽ⁱ⁾ lacking CD8+ T cell, the optimization algorithm searched for a perturbation δ⁽ⁱ⁾ such that classifier f predicted the perturbed patch xₚ⁽ⁱ⁾=x₀⁽ⁱ⁾+δ⁽ⁱ⁾ as having T cells, hence referred to as a counterfactual instance. Ideally, the perturbation should also be minimal and prototypical, meaning it only required targeting a small number of molecule and the counterfactual instance was not far from typical examples of T-cell patches in the training data, to ensure confidence in the model's prediction. A perturbation δ⁽ⁱ⁾ with these desired properties can be obtained by solving the following optimization problem,

$${δ}^{( i )} = {min}_{δ} {L}_{pred} ( {x}_{0}^{( i )} , δ ) + {L}_{dist} ( δ ) + {L}_{proto} ( {x}_{0}^{( i )} , δ ) , \qquad ( 4 )$$

such that

L_{pred}(x₀⁽ⁱ⁾,δ)=cmax(−f(x₀⁽ⁱ⁾+δ),−p),

L_{dist}(δ)=β∥δ∥₁+∥δ∥₂²,

Lₚᵣₒₜₒ(x₀⁽ⁱ⁾,δ)=θ∥x₀⁽ⁱ⁾−δ−proto∥₂², (5)

where δ⁽ⁱ⁾ is a 3D tensor that describes perturbation made to each pixel of the patch and p is the classification threshold for the trained classifier.

The three loss terms in Equation (4) each correspond to a desirable property of the perturbation to discover. The L_{pred} term encouraged the perturbation to increase the classifier's predicted probability of T cells to be larger than p, meaning the network would predict the perturbed tissue patch as having T cells when it previously did not contain T cells. Next, the elastic net regularization term, L_{dist}, minimized the distance between the original patch x₀⁽ⁱ⁾ and the perturbed patch xₚ⁽ⁱ⁾=x₀⁽ⁱ⁾+δ to generate sparse perturbations, favoring strategies that did not require making as many changes to the TME. Lastly, proto⁽ⁱ⁾ in Lₚᵣₒₜₒ refers to, among all patches in the training set that was classified as having T cells, the nearest neighbor of x₀⁽ⁱ⁾. Thus, this last term explicitly guided the perturbed image xₚ⁽ⁱ⁾ to fall in the distribution of training images predicted to contain T cells, leading to perturbed patches that appeared similar to what had been observed in TMEs infiltrated by T cells.

Since drug treatments cannot act at the spatial resolution of individual micron-scale pixels, the search space was constrained to only perturbations that affected all cells in the image uniformly. Specifically, only perturbations that change the level of any molecule by the same relative amount across all cells in an image were searched for. This constraint was incorporated by defining δ⁽ⁱ⁾ in the following way,

$${δ}^{( i )} = {γ}^{( i )} {⊙}_{3} {x}_{0}^{( i )} , \qquad ( 6 )$$

where γ⁽ⁱ⁾≥−1 is a c-dimensional vector with c being the number of protein channels. In practice, γ⁽ⁱ⁾ was directly optimized, where γⱼ⁽ⁱ⁾ can be interpreted as the relative change to the mean intensity of the j-th channel. However, given that the classifier did have fine spatial resolution, targeted therapies such as perturbing only a specific cell type or restricting the perturbation to specific tissue locations can be searched for by changing Equation (6) to match these different types of perturbation.

Taken together, the algorithm obtained an altered image predicted to contain T cells from an original image which lacked T cells, by minimally perturbing the original image in the direction of the nearest training patch containing T cells until the classifier predicted the perturbed image to contain T cells. Since this strategy can find different perturbations for different tumor patches, the set of patch-wise perturbations {δ⁽ⁱ⁾}ᵢ was reduced to a whole-tumor perturbation by taking the median across the entire set.

FIG. 1A-FIG. 1C show non-limiting exemplary workflows of self-supervised training of T cell localization classifier and counterfactual optimization of tissue perturbation. FIG. 1A depicts an overview of Morpheus, a counterfactual optimization framework, for discovering therapeutic strategies predicted to support CD8+ T cell infiltration in human tumors. Morpheus consisted of first training a neural network classifier to predict the presence of CD8+ T cells from multiplexed tissue images where CD8+ T cells were masked. FIG. 1B depicts an overview of the self-supervised training of T cell localization classifier. FIG. 1C depicts the counterfactual optimization of tissue perturbation. Specifically, the trained classifier was used to compute an optimal perturbation vector δ⁽ⁱ⁾ per patch by jointly minimizing three loss terms (L_{pred}, L_{dist}, Lₚᵣₒₜₒ). The perturbation δ⁽ⁱ⁾ represented a strategy for altering the level of a small number of signaling molecules in patch x₀⁽ⁱ⁾ in a way that increased the probability of T cell presence as predicted by the classifier. The optimization also favored perturbations that shifted the image patch to be more similar to its nearest T-cell patches in the training data, shown as proto. Each perturbation corresponded to adjusting the relative intensity of each imaging channel. Finally, taking the median across all such perturbations produced a whole-tumor perturbation vector that represented a potentially effective therapeutic strategy, which was assessed by perturbing in silico tumor images from a test patient cohort and examining the predicted T cell distribution after perturbation.

### Convolutional Neural Networks Trained on IMC Images Accurately Predict T-Cell Distribution From Tumor Signaling Environment

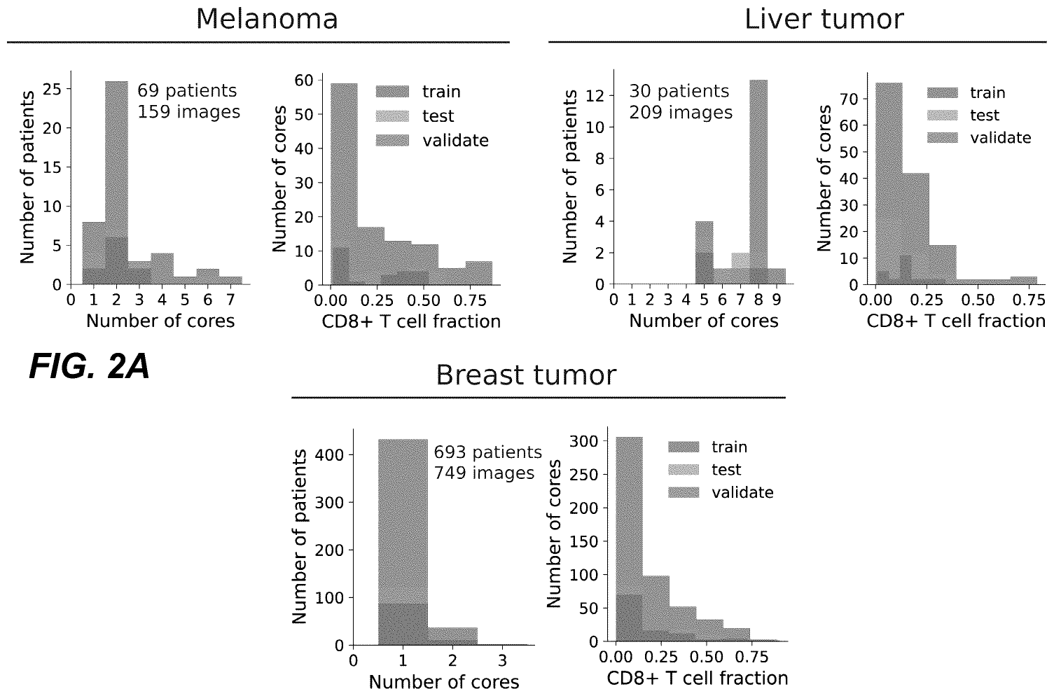

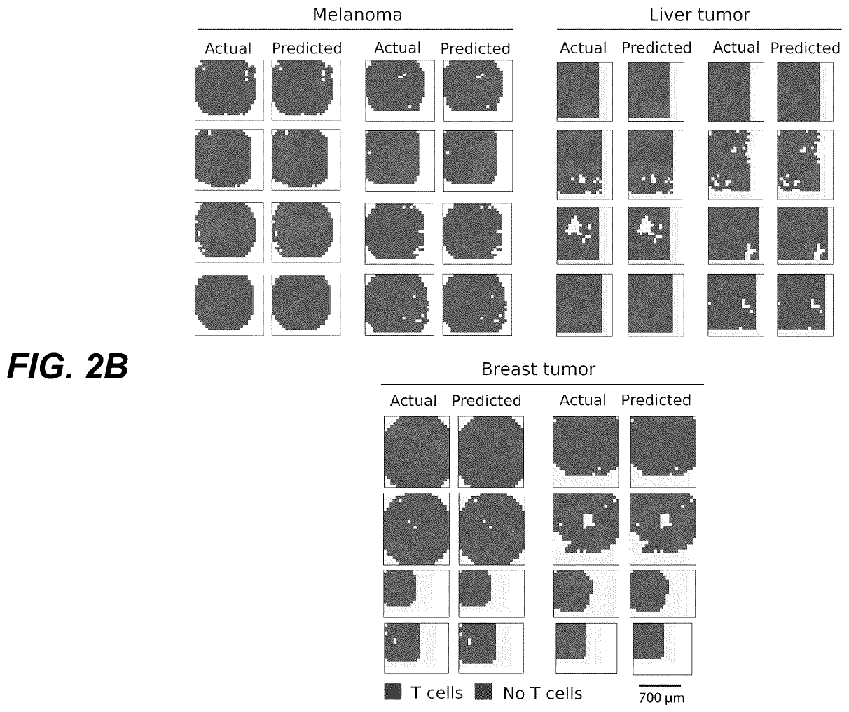

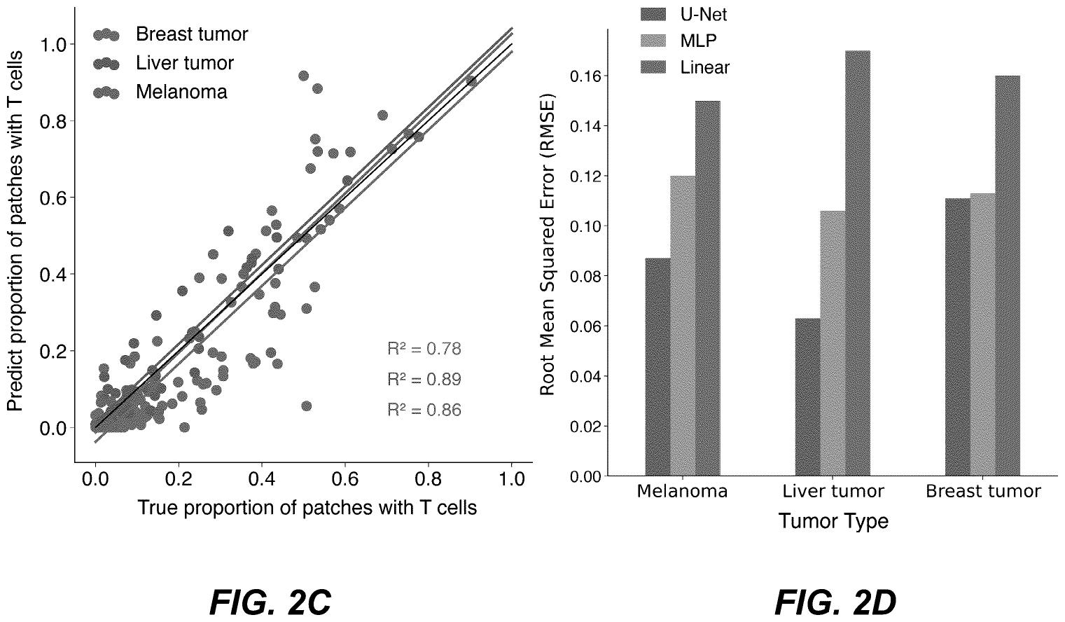

Morpheus was applied to three publicly available IMC datasets of tumors from patients with metastatic melanoma, colorectal cancer (CRC) with liver metastases and breast cancer. For the breast cancer dataset, counterfactual optimization was not applied because the data largely profiled cell type markers, which primarily acted as identifiers rather than functional modulators of T cell infiltration (FIG. 2A). The melanoma dataset was obtained by IMC imaging of 159 tumor cores from 69 patients with stage III or IV metastatic melanoma with samples collected from sites including skin and lymph-node. Each tissue was approximately 1 mm in diameter, imaged at 1 μm resolution across 41 channels, consisting of protein markers for tumor cells, immune cells, stromal cells and 11 different chemokines. The CRC data set consisted of 209 tissue sections taken from 30 patients, including 60 sections from primary CRC tumors, 89 sections from CRC metastases to the liver and 60 “healthy” liver sections obtained away from the metastases. The breast cancer dataset was obtained by IMC imaging of 749 breast tumor cores from 693 patients from the METABRIC study with different molecular sub-types of breast cancer. Most tissues (93%) were in 0.6 mm diameter, image at 1 μm resolution across 37 channels, consisting of markers for tumor, lymphoid, myeloid and stromal cells.

For each of the three tumor datasets, data splitting was performed and a separate U-Net classifier was trained that effectively predicted CD8+ T cell infiltration level in unseen tumor sections. The two classifiers trained on melanoma and CRC data sets achieved the best performance with an AUROC of 0.77 and 0.8 respectively, whereas the classifier trained on breast tumors achieved a AUROC of 0.71. FIG. 2B shows examples of actual and predicted T cell distributions in tumor sections. For each tissue section of a cancer type, the predictions were obtained by applying the corresponding U-Net classifier to each image patch independently. By visual inspection, the classifiers consistently captured the general distribution of T cells. Comparing the true proportion of patches in a tissue section with T cells against the predicted proportion also showed strong agreement (FIG. 2C). The true proportion of patches with T cells was calculated by dividing the number of patches within a tissue section that contained CD8+ T cells by the total number of patches within that section. The performance of the U-Nets was quantified on the entire test data set using the RMSE (Equation (3)), which represented the mean difference between the predicted proportion and the true proportion per tumor section (FIG. 2D). The classifiers performed well on liver tumor and melanoma, achieving a RMSE of only 6% and 8% respectively and a relatively lower performance of 11% on breast tumor. Taken together, these results suggested that the classifier can accurately predict the T cell infiltration status of multiple tumor types.

In order to gain insights into the relative importance of non-linearity and spatial information in the performance of the U-Net on the T cell classification task, the performance of the U-Net was compared to two other models: (A) a logistic regression model (LR) and (B) a multi-layer perceptron (MLP). Both the LR model and MLP model were given only mean channel intensities as input. Thus, neither of the LR model and MLP model had explicit spatial information. Furthermore, the LR model was a linear model with a threshold whereas the MLP was a non-linear model. FIG. 2D shows that across all three cancer datasets, the MLP classifier consistently outperformed the logistic regression model, reducing RMSE by 20-40% to suggest that there were significant nonlinear interactions between different molecular features in terms of their effects on T cell localization. The importance of spatial features on the T cell prediction task, however, was less consistent across cancer types. FIG. 2D shows that for the prediction task in breast tumor, the U-Net model offered negligible boost in performance relative to the MLP model (<2% RMSE reduction), whereas for liver tumor, the U-Net model achieved a RMSE 50% lower compared to the MLP model. This result suggested that the spatial organization of signals can have a stronger influence on CD8+ T cell localization in liver tumor compared to breast tumor or that the functional roles of signals profiled in the liver data sets were more spatially-dependent.

FIG. 2A-FIG. 2D illustrate non-limiting exemplary performance of U-Net classifier trained on IMC images of melanoma, metastatic liver tumor, and breast tumor that accurately predicted T cell distribution. FIG. 2A depicts histograms showing the distribution of tumor cores per patient and CD8+ T cell fractions per core across all three tumor data sets. FIG. 2B depicts predicted and actual T cell distribution of tissue sections from test cohorts in melanoma, liver tumor, and breast tumor data sets. FIG. 2C depicts predicted and true proportion of patches with T cells within a tissue section. Each dot corresponds to a tissue section. Points along the diagonal line indicated perfect prediction. FIG. 2D depicts the RMSE across all (test) tissue sections for three different classes of models.

### Optimization Framework Predicts That Combinatorial Chemokine Treatment can Improve T Cell Infiltration in Melanoma Patients

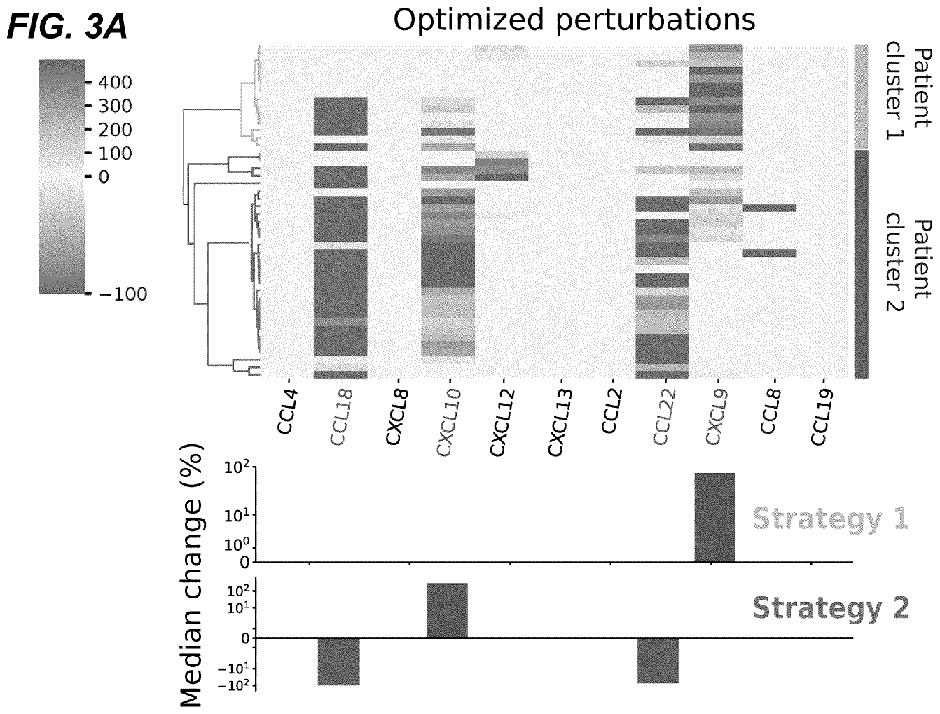

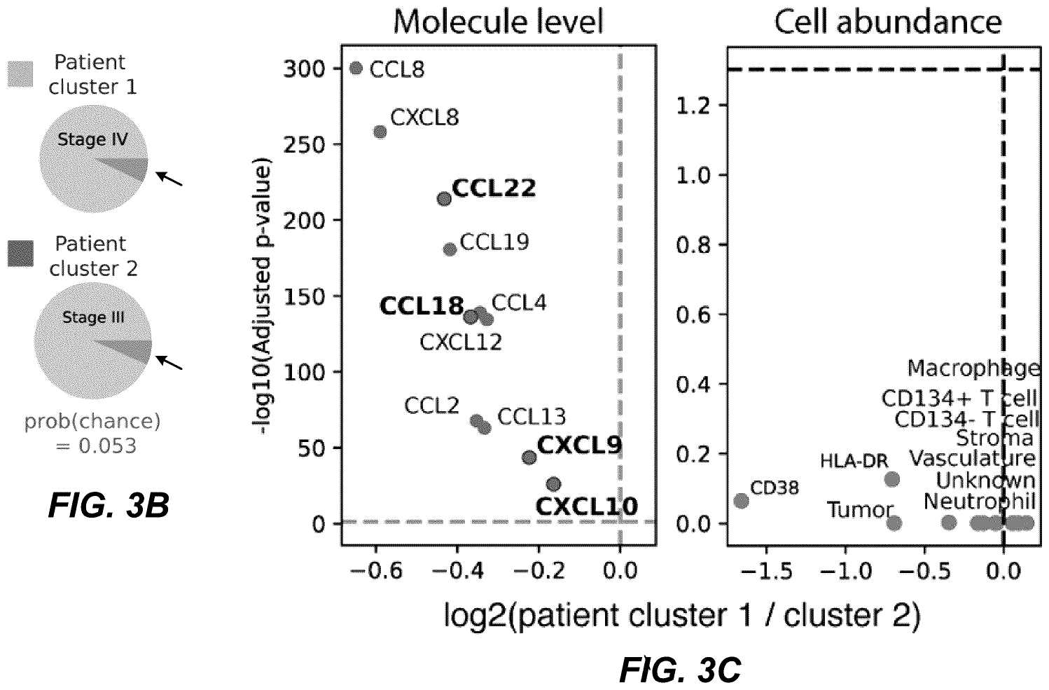

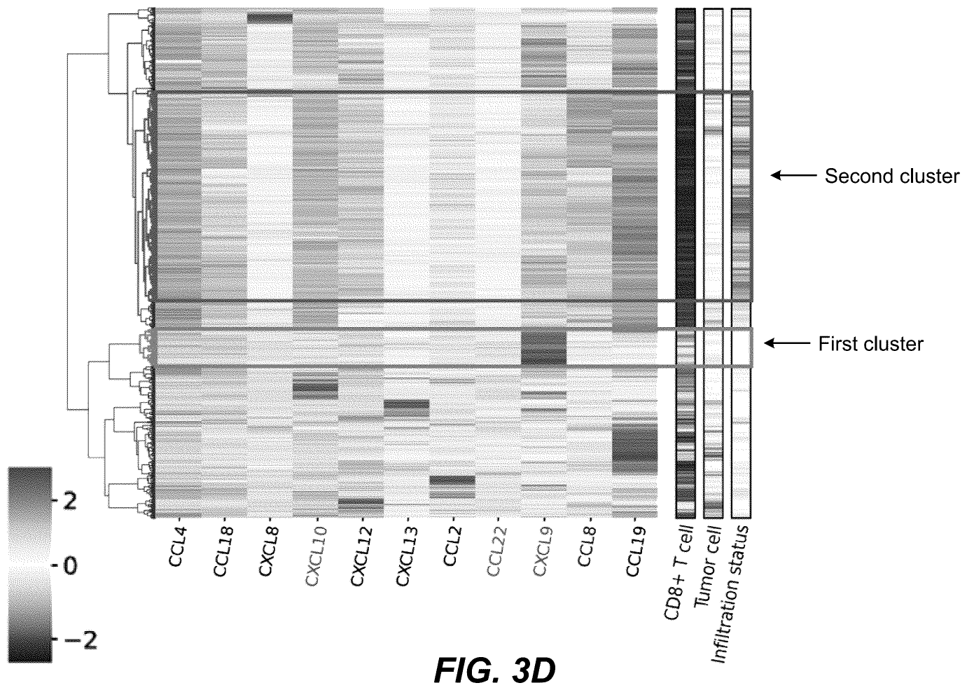

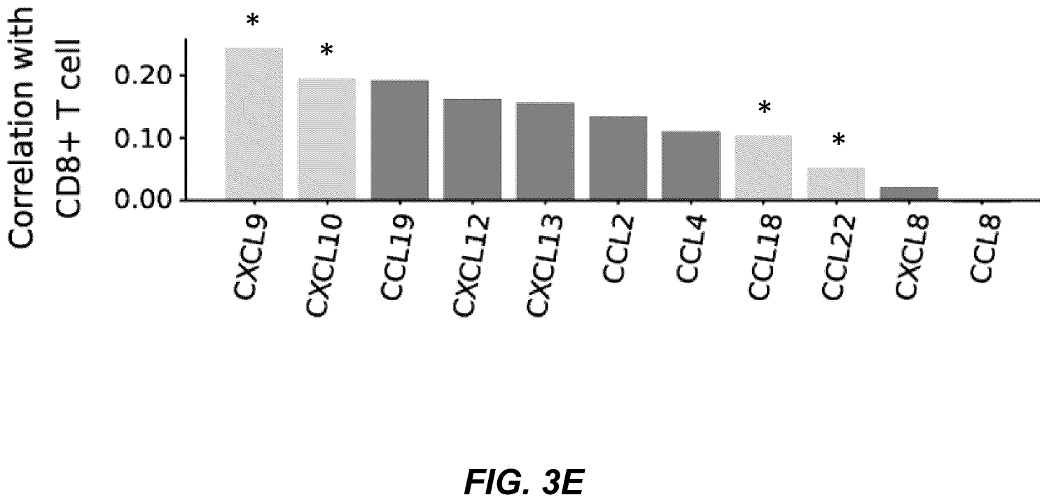

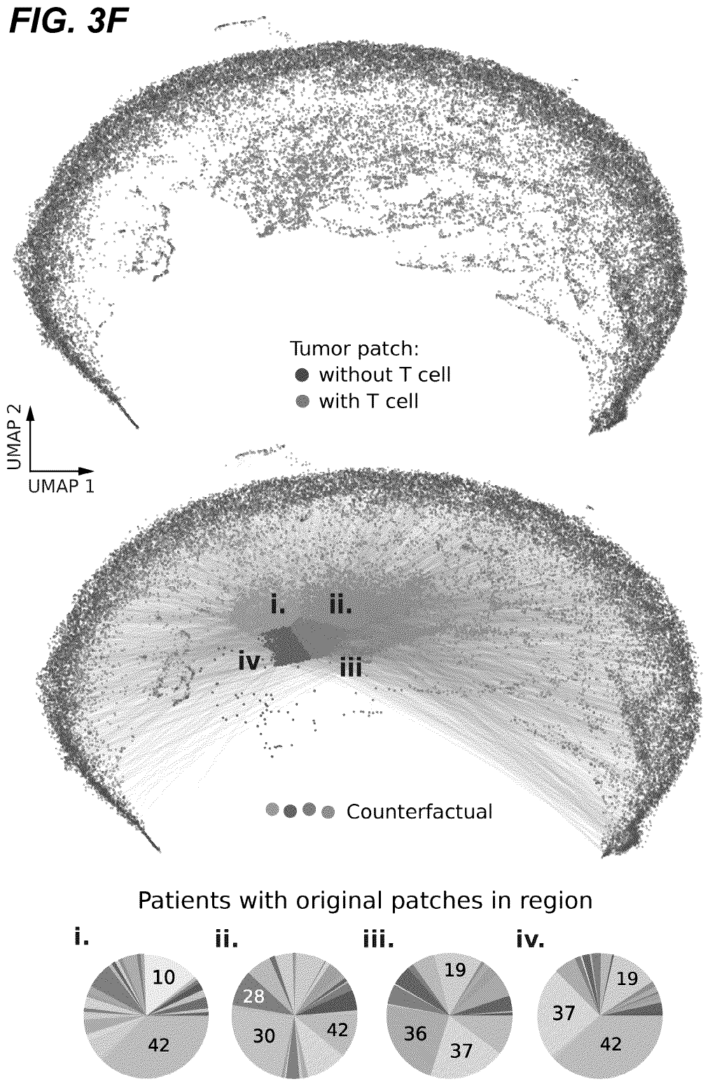

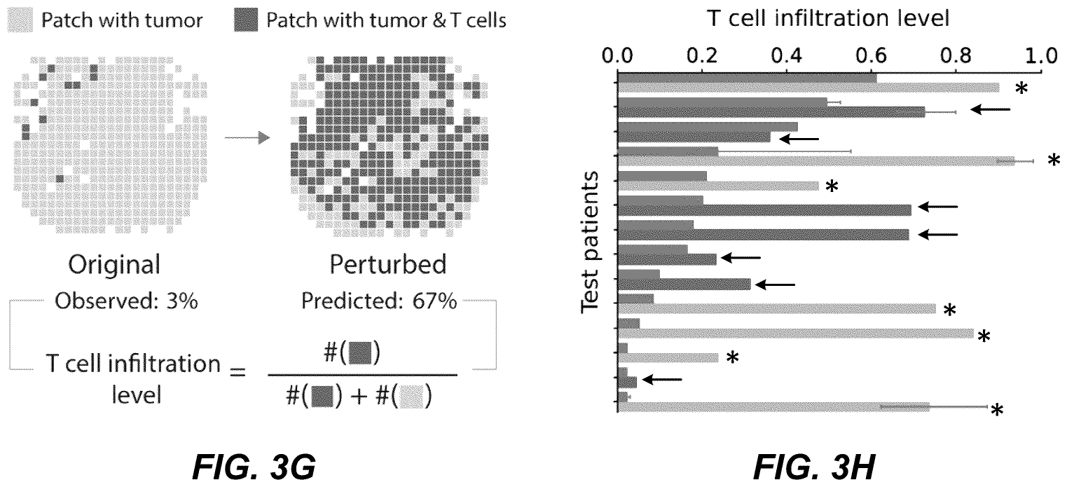

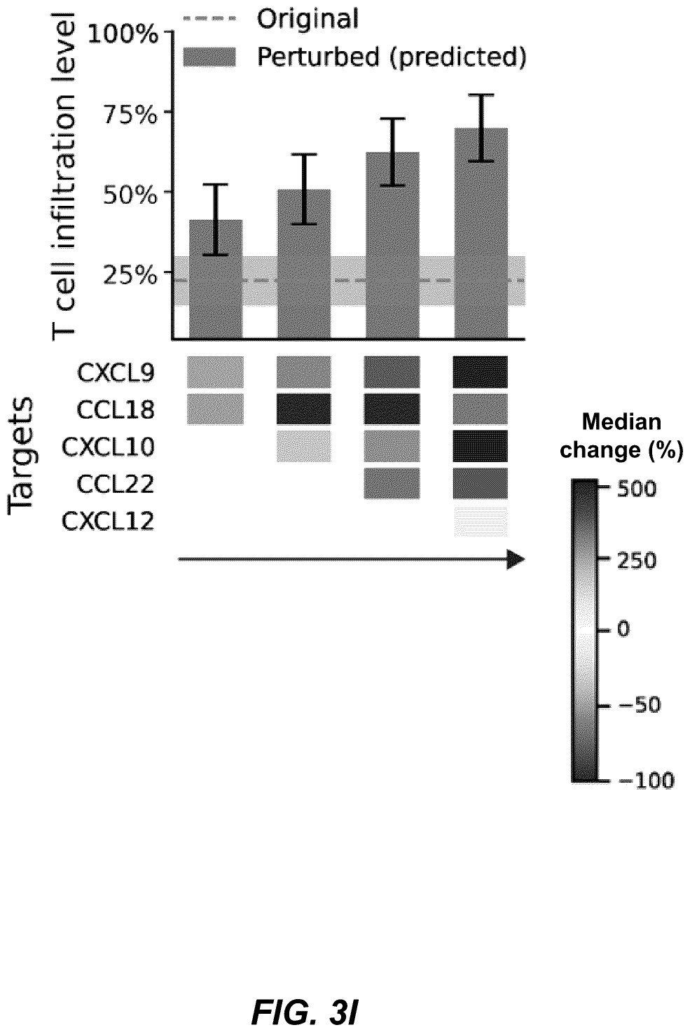

Applying the counterfactual optimization procedure using the U-Net classifier trained on melanoma IMC images, a combinatorial therapy predicted to be highly effective in improving T cell infiltration in melanoma patients was discovered. For simplicity, the optimization algorithm was restricted to only perturbing the level of chemokines, which are a family of secreted proteins that are known for their ability to stimulate cell migration and have been shown to play a significant role in T cell localization in tumors. FIG. 3A shows that patients from the training cohort were separated into two clusters, based on the optimal perturbations computed for each patient. In patient cluster 1, the predicted optimal perturbation was to increase CXCL9 level by 380%, whereas in patient cluster 2, the optimal strategy consisted of increasing CXCL10 level by 280% and decreasing CCL18 and CCL22 levels by 100% and 70% respectively (FIG. 3A). Both CXCL9 and CXCL10 are well-known for playing a role in the recruitment of CD8+ T cells to tumors. On the other hand, CCL22 is known to be a key chemokine for recruiting regulatory T cells and CCL18 is known to induce an M2-macrophage phenotype, and their expression likely promotes an immunosuppressive microenvironment inhibitory to T cell infiltration and function.

FIG. 3B shows that the choice of which of these two strategies was selected for a patient appeared to be strongly associated with the patient's cancer stage, with strategy 1 being significantly enriched for stage IV metastatic melanoma patients and strategy 2 being significantly enriched for stage III patients, with a probability of 0.053 of such difference being due to chance. Probing deeper into the difference between these two patient clusters, all chemokines had lower mean expression in the tumors of patients in cluster 1 compared to cluster 2, while there were no significant differences between the two groups in terms of the cell type compositions within tumors (FIG. 3C). The fact that the levels of CCL22 and CCL18 were 37% and 31% higher in patients from cluster 2 and that both chemokines had been implicated in the recruitment of immunosuppressive cells, offered one possible explanation as to why the optimization algorithm suggested inhibiting CCL18 and CCL22 only for patients in cluster 2. However, the switch from boosting CXCL9 to CXCL10 was not as straightforward, suggesting possible interaction effects among chemokines such that boosting CXCL10 was an important supplement to blocking CCL18 and CCL22.

The optimization procedure selected perturbations that would make the chemokine composition of a TME more similar to T cell rich regions of immune-infiltrated tumors. FIG. 3D shows that melanoma tissue patches can be clustered into distinct groups based on their chemokine concentration profile. The first cluster contained exactly the patches from immune-infiltrated tumors that contained both tumor and T cells, which likely represented a chemokine signature that was suitable for T cell infiltration. On the other hand, a second cluster, which contained patches from immune-desert tumors that have tumor cells but no T cells, likely represented an unfavorable chemokine signature. In comparison to the second cluster, FIG. 3D shows the first cluster contained elevated levels of CXCL9, CXCL10 and reduced levels of CCL22, which partially agreed with the perturbation strategy (FIG. 3A) discovered by the optimization procedure. Lastly, FIG. 3E shows that the four selected chemokine targets cannot simply be predicted from correlation of various chemokine levels with the presence of CD8+ T cells, as both CCL18 and CCL22 were weakly correlated (<0.1) with CD8+ T cells even though the optimized perturbations required inhibiting both chemokines, suggesting the presence of significant nonlinear effects not captured by correlations alone.

How the optimization procedure searches for efficient perturbations can be directly observed by viewing both the original patch and perturbed patches in a dimensionally-reduced space. FIG. 3F shows a UMAP projection where each point represents the chemokine profile of an IMC patch. Patches with CD8+ T cells were well-separated from those without CD8+ T cells. The colored sections in the top panel of FIG. 3F illustrates the perturbation for each patch as computed by the optimization algorithm, demonstrating two key features of the algorithm disclosed herein. First, optimized perturbations pushed patches without T cells towards the region in UMAP space occupied by T-cell-infiltrated patches. Second, the sections in FIG. 3F were colored to show that optimized perturbations seemingly efficient in that patches were perturbed just far enough to land in the desired region of space. Specifically, points indicating without T cells that started out on the right edge ended up closer to the right after perturbation in points labeled ii or iii, while points that start on the left/bottom edge end up closer to the left (i)/bottom (iv), respectively. This observation was made while noting that UMAP, though designed to preserve the topological structure of the data, was not a strictly distance-preserving transformation. Furthermore, the pie charts (i-iv) were colored by the patient of origin to show the region of space where points being perturbed to were not occupied by tissue samples from a single patient with infiltrated tumor. Rather, these regions consisted of tissue samples from multiple patients, suggesting that the optimization procedure can combine information from different patients when searching for therapeutic strategies.

After applying the second set of perturbations from FIG. 3A in silico to IMC images of a tumor, FIG. 3G shows that T cell infiltration level (defined as the proportion of tumor patches with T cells) was predicted to increase by 20 fold. Two strategies were performed on patients in the test cohort in silico after stratifying by stage, using perturbation strategy 1 for patients with stage IV melanoma and perturbation strategy 2 for patients with stage III melanoma. FIG. 3H shows that this predicted improvement held across nearly all 14 patients from the test group, boosting T cell infiltration level from an average of 23% across tumor sections to a predicted 63% post perturbation. For the three test patients with multiple tumor sections (patients 64, 57, 89), little variation in predicted improvement was seen across samples.

The combinatorial nature of the designed perturbation strategy was crucial to its predicted effectiveness. The importance of combinatorial perturbation was systematically explored by changing parameter β of Equation (4) which adjusts the sparsity of the solution, where a sparser solution means fewer molecules are perturbed. FIG. 3I shows that perturbing multiple targets was predicted to be necessary for driving significant T cell infiltration across multiple patients, with the best perturbation strategy involving two targets predicted to generate only 60% of the infiltration level achieved by the best perturbation strategy involving four targets. In conclusion, within the scope of the chemokine targets considered, combinatorial perturbation of the TME appeared necessary for improving T cell infiltration in metastatic melanoma.

FIG. 3A-FIG. 3I illustrate non-limiting exemplary combinatorial chemokine therapy predicted to drive T cell infiltration in groups of metastatic melanoma patients. FIG. 3A depicts whole-tumor perturbations optimized across IMC images of individual patients (row) from the training cohort, with bar graph showing the median relative change in intensity for each molecule. FIG. 3B depicts distribution of cancer stages among patients within two clusters and chance probability from hypergeometric distribution. Arrows point to sections of unknown stage. FIG. 3C depicts volcano plot comparing chemokine level and cell type abundance from patient cluster 1 and 2, computed using mean values and Wilcoxon rank sum test. The dots at the bottom of the right chart indicate no statistical significance. FIG. 3D depicts patch-wise chemokine profile (left) and 1-D heatmap (right). The 1-D heatmap (right) includes: infiltration status (light/dark=infiltrated/deserted tumor), tumor cell (light/dark=present/absent), CD8+ T cells (light/dark=present/absent). FIG. 3E depicts correlation between the chemokine signals and the presence of CD8+ T cells in a patch. Chemokines discovered as key perturbations are marked with *. FIG. 3F depicts UMAP projection of patches from tumor images (chemokine channels only) and connections of UMAP projection of patches without T cells and their corresponding perturbed images (i.e., counterfactual instance). The sections marked as i, ii, iii, and iv corresponds to k-nearest neighbor clusters of the perturbed images. Pie charts show the distribution of patients whose tumor images were found near the four clusters (i-iv) of perturbed images. FIG. 3G depicts cell maps computed from a patient's IMC image, showing the predicted distribution of T cells before and after perturbation. FIG. 3H depicts original vs. perturbed (predicted) mean infiltration level across all patients (test cohort) with 80% confidence interval (CI) (only shown for patients with more than 2 tumor sections). Stage IV patients received perturbation strategy 1 are marked with *, and stage III patients received perturbation strategy 2 are marked with arrows. FIG. 3I depicts mean (predicted) infiltration level across all patients (test cohort) for different perturbation strategy of varying sparsity, with error bar represents 95% CI.

### Optimization Framework Predicts Two Patient-Dependent Therapeutic Strategies for Driving T Cell Infiltration in CRC Patients With Liver Metastases

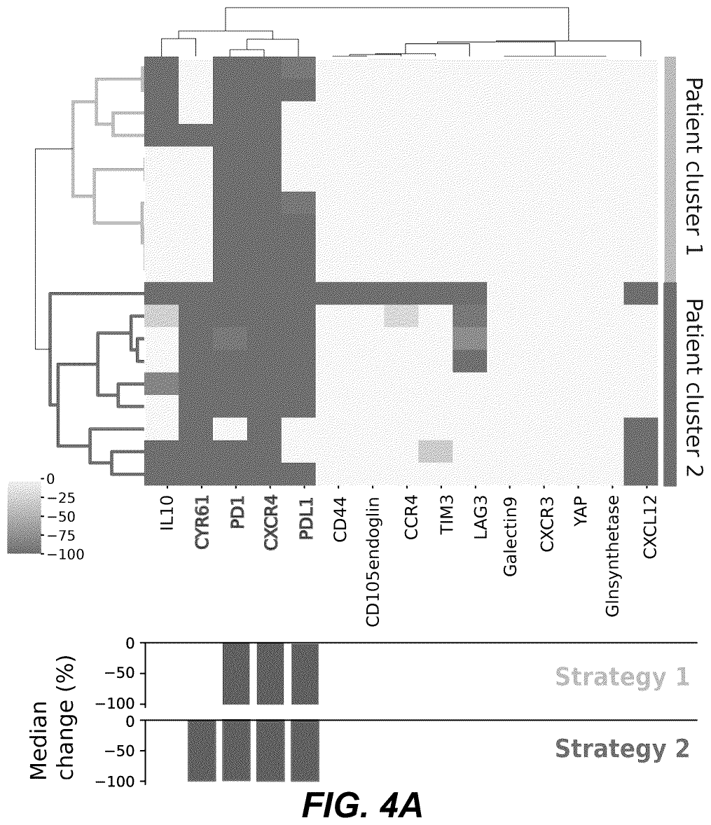

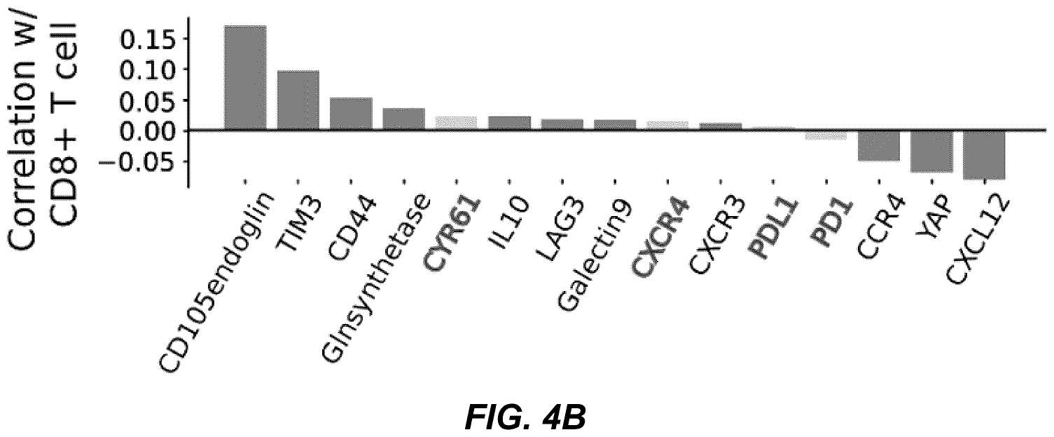

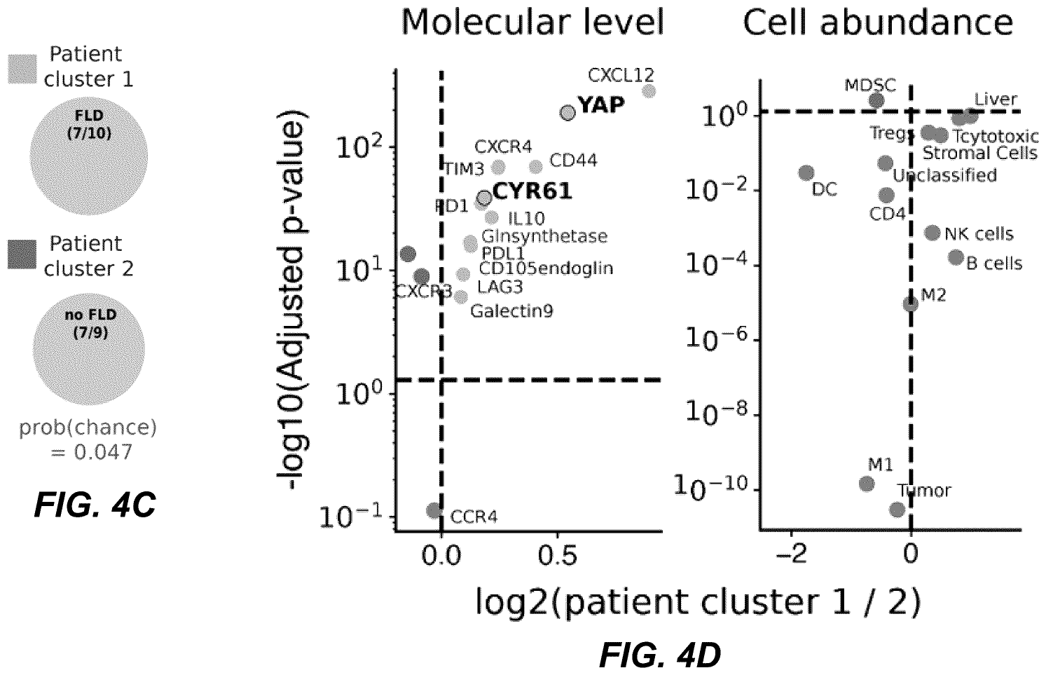

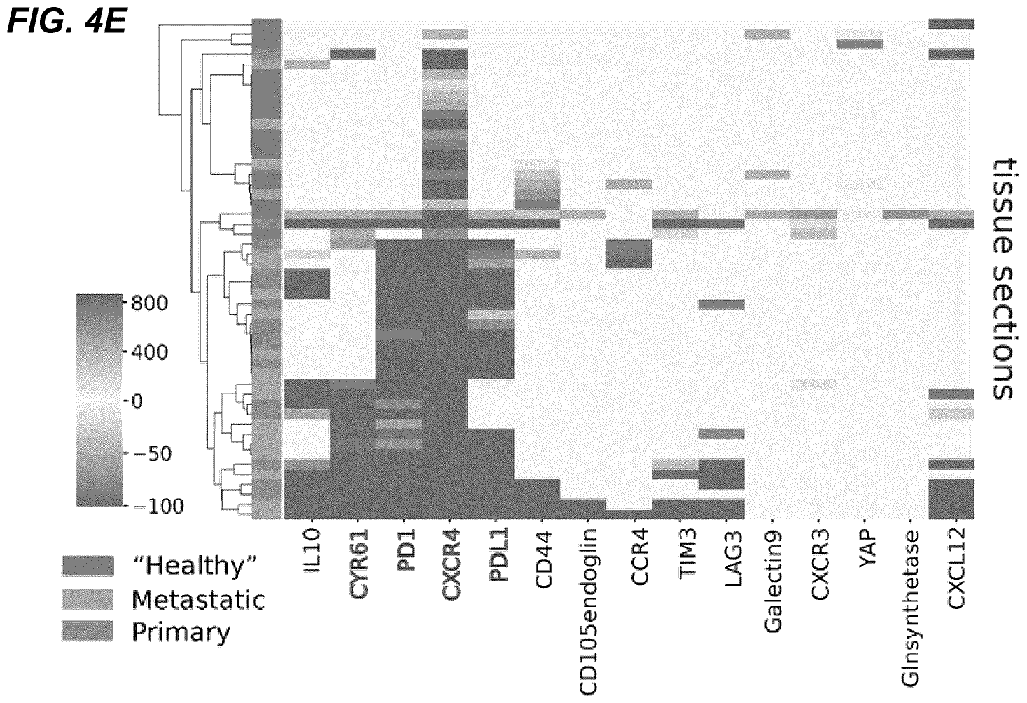

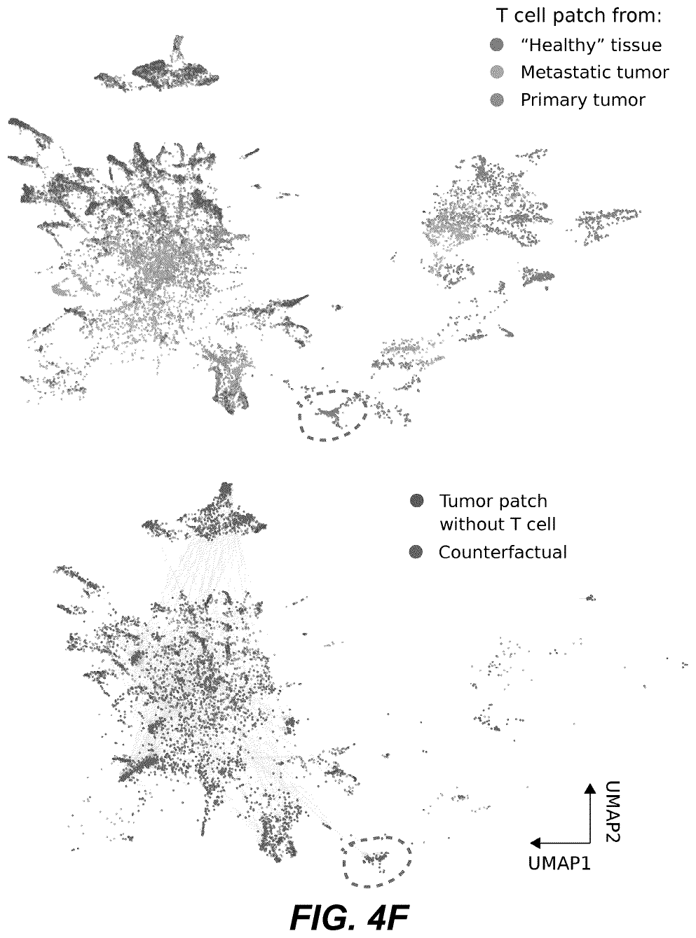

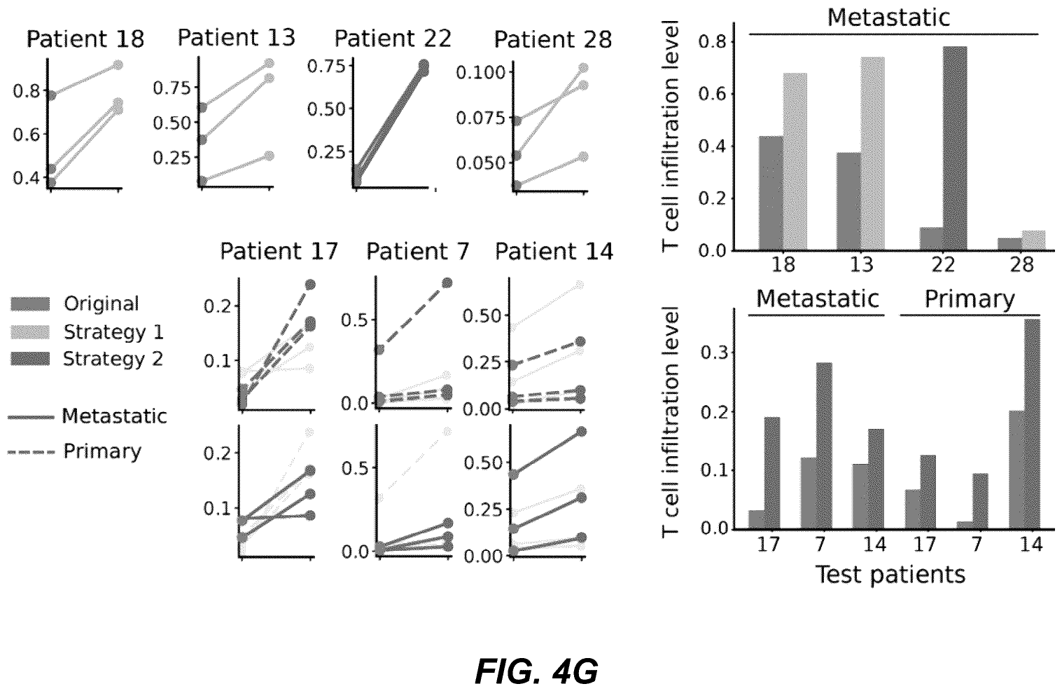

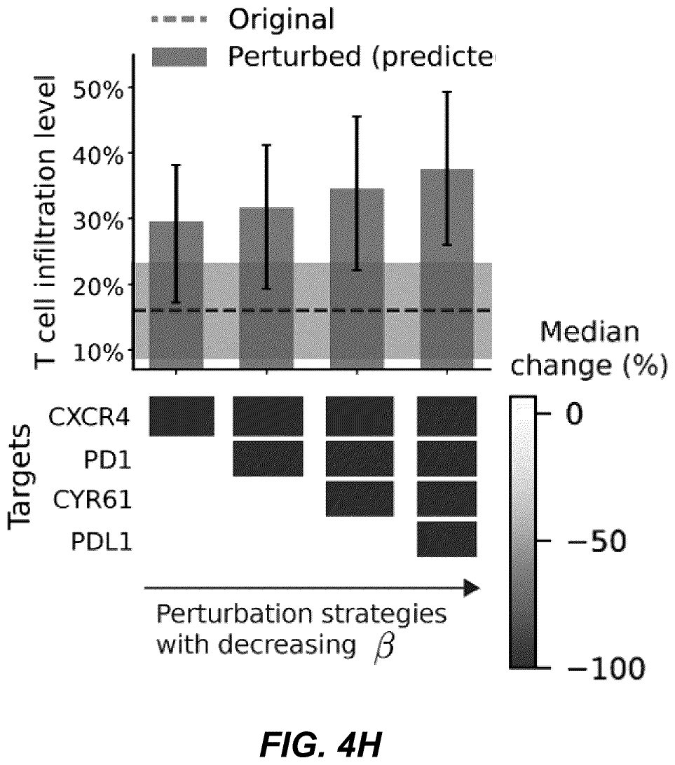

Applying Morpheus to IMC images from the CRC cohort, two patient-dependent therapies were predicted to be highly effective in improving T cell infiltration. FIG. 4A shows the optimal perturbations computed for every patient from the training cohort, aggregated over all primary tumor and liver metastases samples for each patient. Hierarchical clustering revealed that the method disclosed herein consistently discovered two patient-dependent strategies for improving T cell infiltration. The optimized strategy for the first cluster of patients involved inhibiting PD-1, PD-L1, and CXCR4. While for the second group of patients, the optimized strategy involved inhibiting CYR61, PD-1, PD-L1, and CXCR4 (FIG. 4A). Interestingly, all four of the perturbation targets correlated poorly with the presence of CD8+ T cells compared to the other proteins that were not selected as perturbation targets (FIG. 4B), suggesting the presence of significant spatial and nonlinear effects not captured by correlations alone.

All perturbation targets identified by the optimization procedure had been found to play crucial roles in suppressing T cell function in the TME, and treating patients with inhibitors against subsets of the selected targets had already improved T cell infiltration in human CRC liver metastases. Regulatory T cells (Tregs) are recruited into tumor through CXCL12/CXCR4 interaction, and the PD-1/PD-L1 pathway inhibits CD8+ T cell activity and infiltration in tumors. In addition, CYR61 is a chemoattractant and is shown to drive M2 TAM infiltration in patients with CRC liver metastases. Inhibition of both PD-1 and CXCR4, which were consistently selected by the algorithm disclosed herein as targets, was shown to increase CD8+ T cell infiltration in both CRC patients and mouse models. Finally, FIG. 4A shows that the fifth most common proposed perturbation involved inhibiting IL-10. Indeed, blocking of IL-10 was recently shown to increase the frequency of non-exhausted CD8+ T cell infiltration in slice cultures of human colorectal cancer liver metastases.

Without being bound by any theories, the emergence of the two distinct perturbation strategies can be explained by variation in liver fat build-up among patients. Patient cluster 1 is made up of significantly more patients with fatty liver disease (70%) compared to patient cluster 2 (22%), where the probability of this due purely to chance is 0.047 (C). Furthermore, FIG. 4D shows that both YAP and CYR61 levels were significantly (50% and 15% respectively) higher in tumors from patient cluster 1 than in tumors from patient cluster 2. Indeed, CYR61 is known to be associated with non-alcoholic fatty liver disease (NAFLD) and YAP is a transcription coregulator that induces CYR61 expression). However, despite patients in cluster 1 having higher levels of CYR61, patients in cluster 2 only had higher levels of CYR61 where the optimal strategy involves blocking CYR61. Without being bound by any theories, this seemingly paradoxical finding can arise because removing CYR61 from patients in cluster 1 represented a more pronounced perturbation, given their inherently higher concentration. A perturbation of this magnitude would likely shift the tumor profile significantly away from the distribution of the training data, where the classifier's prediction about the perturbation's effect became less reliable. Thus, such a perturbation would be heavily penalized by the Lₚᵣₒₜₒ term in Equation (5).

The method disclosed herein discovered tissue-dependent perturbation strategies without needing any additional metadata beyond the image patches (FIG. 4E). As depicted in FIG. 4E, by aggregating perturbations at the individual tissue level, the optimized perturbation for “healthy” liver sections was straightforward, necessitating only the inhibition of CXCR4. In contrast, promoting T cell infiltration into primary colon tumors was anticipated to involve targeting a minimum of three signals. The method disclosed herein revealed that liver metastases appeared to fall between these two tissue types, as the optimal proposed perturbation for some of the liver metastasis samples was to block CXCR4, while the rest required the inhibition of the same set of signals as primary tumors. Furthermore, direct comparison between perturbations optimized for metastatic tumor and primary tumor samples did not reveal a significant difference in strategy. The discrepancy between tissues can partly be understood by plotting a UMAP projection of all T cell patches from the three tissue types (FIG. 4F, left). The clear separation between T cell patches from “healthy” tissue and those from primary tumors underscored that the signaling compositions driving T cell infiltration likely differed substantially between the two tissue types. This distinction was likely what prompted the method disclosed herein to identify markedly different perturbation strategies. Furthermore, some patches from metastatic tumors co-localized with “healthy” tissue patches in UMAP space, while other patches co-localized with primary tumor patches. This observation again aligned with previous results, where optimized perturbations for various metastatic tumor samples bore similarities to both “healthy” tissue and primary tumor strategies (FIG. 4E).

Despite the CRC dataset comprising a complex blend of healthy, tumor, and hybrid metastatic samples, the method disclosed herein naturally targeted the most pertinent tissue types when optimizing perturbations. During both the model training and counterfactual optimization phases, the three tissue types were not specifically segregate. Furthermore, tissue type labels or any additional metadata were not provided. Despite these nuances, FIG. 4F shows that the counterfactual instances for tumor patches from primary and metastases samples were mostly perturbed to be near T cell patches from primary and metastatic tumor, instead of being perturbed to be similar to T cell patches from “healthy” tumors. This was a direct consequence of the prototypical constraint, which encouraged patches to be perturbed towards the closest positive (containing T cell) instance. For a tumor patch without T cells from a metastatic tumor, the closest positive patch was more likely to be a tumor patch with T cell from a metastatic tumor than to be a T cell patch from a “healthy” tissue. However, there were occasional exceptions where T cell patches from “healthy” tissues could influence the optimization of tumor tissues, as outlined by the dashed ellipse in FIG. 4F, especially if they shared features similar to those of tumor regions.

The two therapeutic strategies discovered were generalized to patients in the test cohort (FIG. 4G and FIG. 4H). Given that one of the two therapeutic strategies enriched for patients with FLD and the other for patients without FLD, different perturbation strategies were applied in silico across all test patients depending on their FLD status. Aggregating to the patient level, FIG. 4G shows that CD8+ T cell infiltration level was predicted to increase substantially for nearly all patients, with the exception of patient 28. Furthermore, aggregating to the entire test cohort, FIG. 4H shows a statistically significant boost to mean infiltration level from 15% to a predicted 35% post perturbation. However, when comparing individual tissue samples, FIG. 4G reveals significant variation in the predicted response to perturbation among samples from the same patient and tissue types. In patient 7, one primary tumor sample was predicted to see a nearly three-fold increase in T cell infiltration after perturbation. However, almost no change was expected for the another two primary and three metastatic samples. Similar patterns were observed in patients 14 and 17. This marked variability in response among a significant portion of test patients underscored the challenges posed by intra-tumor and inter-patient heterogeneity in devising therapies for CRC with liver metastases. This result further implied that, for studying CRC with liver metastases, collecting numerous tumor sections per patient could be as crucial as establishing a large patient cohort. Lastly, combinatorial perturbation was again predicted to be necessary to drive significant T-cell infiltration in large patient cohorts. By increasing β in Equation (4), strategies with between one and four (total) targets were generated, where four-target perturbation was the only strategy predicted to produce a statistically significant boost to T-cell infiltration (FIG. 4H).

FIG. 4A-FIG. 4H show non-limiting exemplary blocking subsets of PD-L1, CXCR4, PD-1, and CYR61 predicted to drive T cell infiltration in CRC cohort. FIG. 4A depicts optimized tumor perturbations aggregated to the patient (row) level (train cohort). Bar graph (FIG. 4A) shows the median relative change in intensity for each molecule across all patients. FIG. 4B depicts correlation between the level of different molecules and the presence of CD8+ T cells in a patch. FIG. 4C depicts pie charts showing proportion of patients in each cluster that have fatty liver disease (FLD), and chance probability from hypergeometric distribution. FIG. 4D depicts volcano plot comparing molecule levels and cell type abundance between the two patient clusters using tumor tissues, computed using mean values and Wilcoxon rank sum test with Bonferroni correction. FIG. 4E depicts optimized perturbations aggregated to the level of tissue samples (row). FIG. 4F depicts UMAP projection of individual IMC patches. Left UMAP shows T cell patches colored by the tissue samples they were taken from. Right UMAP shows counterfactual (perturbed) instances optimized for tumor patches without T cells. FIG. 4G depicts line plots showing T-cell infiltration level for each tissue section from the test cohort, before and after perturbation; and bar plots showing predicted mean T-cell infiltration level in test cohort. FIG. 4H depicts mean predicted infiltration level across all test patients using perturbation strategies of varying sparsity, obtained by varying β in Equation (4), with error bar represents 95% CI.

### Discussion

The deep learning framework disclosed herein, Morpheus, combined deep neural networks with optimization methods from explainable AI to directly predict therapeutic interventions.

In some embodiments, Morpheus can be readily scale to deal with large diverse sets of patients' samples including metachronous tissue from the same patients but different sites, which would be crucial as more spatial transcriptomics and proteomics datasets are quickly becoming available. Larger data sets could allow the training of more complex models such as vision transformers, capturing long range interactions in tissue profiles to be more predictive of T cell localization. Furthermore, a large set of diverse patient samples would more accurately capture the extent of tumor heterogeneity, enabling Morpheus to discover therapeutic strategies for different sub-classes of patients.

In some embodiments, Morpheus can be applied to spatial transcriptomics data sets with hundreds to thousands of molecular channels. Although spatial transcriptomics can profile significantly more molecules compared to current spatial proteomic techniques, the number of spatial transcriptomic profiles of human tumors can be limited due to the cost, with most public data sets containing single tissue sections from 1-5 patients, which can be far too small for the present application. However, spatial transcriptomics can likely be more standardized compared to proteomics, which use customized panels. As commercial platforms for spatial transcriptomics start to come online, large scale spatial transcriptomics data sets can be made available, with 70-90% of the same probes shared between experiments.

Generalized therapies can be identified by pooling predictions across multiple patient samples. However, Morpheus can also be applied to find personalized therapy for treating individual patients. The variation in the optimized perturbations observed among patients in both melanoma and liver data sets suggested personalized treatments could be significantly more effective compared to generalized therapies (FIG. 3A and FIG. 4A). Furthermore, FIG. 4G shows that a therapeutic strategy could have highly variable effects across different tissue samples from the same patient. This variability suggested that in order to generate therapy for individual patients, a large set of tissue sections from the patient's tumor can be needed to have a representative sample. The optimization procedure disclosed herein can then be applied to a random subset of the samples, and then the resulting perturbation strategy can be tested on the remaining samples to see how well the strategy is predicted to perform across an entire tumor or other primary/secondary tumors.

Morpheus can be incorporated in a closed loop with experimental data collection. Data can be collected from patients or animal models with perturbed/engineered signaling context. This data can be easily fed back into the classifier model to refine the model's prediction. The perturbation could be based on what the model predicts to be effective interventions, as is the case with Morpheus. Tissue samples can also be studied, on which the model tends to make the most mistake and the model can be trained specifically using samples from similar sources, such as the same patient or disease state.

Cell-type specific perturbations can also be incorporated into Morpheus, which can be done by directly restricting the optimization to only alter signals within specific cell types. Experimentally, the therapeutic predictions in animal models of melanoma and CRC liver metastases can be tested.

### Methods

### Data Split

For all three IMC data sets, the same data splitting scheme was followed to divide patients into three different groups (e.g., training, validation and testing), while ensuring similar class balance across the groups. As used here, the term “class balance” means that the proportion of image patches with and without T cells were roughly equal across the three groups. Specifically, each image within a data set was divided into 48 μm×48 μm patches and the number of patches with and without CD8+ T cells was computed for each image. Furthermore, each patch was down-sampled from 48×48 pixels to 16×16 pixels dimension where each pixel represented a 3 μm×3 μm region. Spectral analysis was applied to study the effect of using different patch sizes to predict T cell infiltration. The selected patch size remained highly informative of T cell presence (see Additional Information section). Next, the patients were shuffled between the three groups until three criteria were met: 1) the number of patients across the three groups followed a 65/15/20 ratio, 2) the difference in class proportion across the three groups was less than 2%, and 3) the training set contained at least 65% of total image patches.

### Melanoma Data Set

Melanoma data set contained 159 images or cores taken from 69 patients. Table below contains summary statistics for the three groups that patients were split into.

TABLE 1
DATA SPLIT STATISTIC FOR MELANOMA IMC DATASET

| | | | Proportion of |
|---|---|---|---|
| Group | Patient count | Patch count | positive patches |
| Training | 102 | 23741 | 29.6% |
| Validation | 28 | 6045 | 30.3% |
| Testing | 29 | 5950 | 30.4% |

### CRC Liver Metastases Data Set

CRC liver metastases dataset contained 209 images or cores taken from 30 patients. Table below contains summary statistics for the three groups that patients were split into.

TABLE 2
DATA SPLIT STATISTIC FOR CRC COHORT IMC DATASET

| Group | Patient count | Patch count | Proportion of positive patches |
|---|---|---|---|
| Training | 19 | 44449 | 15.9% |
| Validation | 4 | 6957 | 14.4% |
| Testing | 7 | 14907 | 15.9% |

### Breast Tumor Data Set

Breast tumor dataset contained 693 images or cores taken from 693 patients. Table below contains summary statistics for the three groups that patients were split into.

TABLE 3
DATA SPLIT STATISTIC FOR
BREAST TUMOR IMC DATASET

| Group | Patient count | Patch count | Proportion of positive patches |
|---|---|---|---|
| Training | 485 | 41104 | 23.7% |
| Validation | 113 | 9015 | 23.4% |
| Testing | 151 | 12987 | 23.8% |

### Classifier Training

In the present disclosure, three classes of models were trained to perform the T cell prediction task. All models disclosed herein were trained with early stopping based on the validation Matthews Correlation Coefficient (MCC) for 10-20 epochs. All models were trained on an NVIDIA GeForce RTX 3090 Ti GPU using PyTorch version 1.13.1.

### T Cell Masking Strategy

The purpose of model training was for the model to learn molecular features of the CD8+ T cell's environment that was indicative of its presence. Thus, it was important to remove features of the image that were predictive of CD8+ T cell presence but were not part of the cell's environment. A non-trivial cell masking strategy was devised to remove T cell-intrinsic features without introducing new features that were highly predictive of T cell presence but were not biologically relevant. A simple masking strategy of zeroing out all pixels belonging to CD8+ T cells would introduce contiguous regions of zeros to image patches with T cells, which could be an artificial feature that is nonetheless highly predictive of T cell presence and hence would likely be the main feature learned by a model during training. To circumvent this issue, a cell “pixelation” step was applied to the original IMC image where each cell was reduced to a single pixel positioned at the cell's centroid. The value of this pixel was the sum of all pixels originally associated with the cell, representing the total signal from each channel within the cell. Then, this “pixelated” image was masked by zeroing all pixels representing CD8+ T cells. Since there were usually at most two T cell pixels in an image patch, zeroing them in a 16×16 pixels image, where most of the pixels (>90%) were already zeroes, was not likely to introduce a significant signal that was predictive of T cell presence. This strategy was effective at masking T cells without introducing additional features through a series of image augmentation experiments (see Additional Information section).

### Logistic Regression Models

A single-layer neural network was trained on the average intensity values from each molecular channel to obtain a logistic regression classifier, predicting the probability of CD8+ T cell presence in the image patch. This model represented a linear model where only the average intensity of each molecule was used for prediction instead of their spatial distribution within a patch.

### MLP Models

Similar to a logistic regression model, the Multilayer Perceptron (MLP) also used averaged intensity as input features for prediction but was capable of learning nonlinear interactions between features. The MLP model consisted of two hidden layers (30 and 10 nodes) with ReLU activation.

### U-Net Models

To train networks that could make full use of the spatial information, a fully convolutional neural network with the U-Net architecture was used. The U-Net architecture consisted of a contracting path and an expansive path, which gave it a U-shaped structure. The contracting path consisted of four repeated blocks, each containing a convolutional layer followed by a Rectified Linear Unit (ReLU) activation and a max pooling layer. The expansive path mirrored the contracting path, where each block contained a transposed convolutional layer. Skip connections concatenated the up-sampled features with the corresponding feature maps from the contracting path to include local information. The output of the expansive path was then fed to a fully-connected layer with softmax activation to produce a predicted probability. The model was trained from scratch using image augmentation to prevent over-fitting, including random horizontal/vertical flips and rotations, in addition to standard channel-wise normalization. The U-Net classifiers were trained using stochastic gradient descent with momentum and a learning rate of 10′ on mini-batches of masked image patches.

### Counterfactual Optimization

Given an IMC patch x⁽ⁱ⁾ without T cells, and a classifier f, the goal was to find a perturbation δ⁽ⁱ⁾ for the patch such that f classified the perturbed patch as having T cells. For CNN models, δ⁽ⁱ⁾∈ [symbol] ^{w×l×d} was a 3D tensor that described changes made for every channel, at each pixel of the patch. For MLP models, δ⁽ⁱ⁾∈ [symbol] ^{d} was a vector where each element represents changes to the average intensity of a channel in patch i. This distinction between the two models highlighted differences in the type of perturbations that each model can discover. The MLP model can find region-wide perturbation such as changing the overall level of an extracellular molecule, whereas the CNN model allowed for cell-type specific perturbations since perturbations were being computed at the level of micron-scale pixels.

Given a CNN classifier f and an IMC patch x⁽ⁱ⁾ such that f(x₀⁽ⁱ⁾)= [symbol] (T cells present)<p, where p>0 is the classification threshold below which the classifier predicts no T-cell, a perturbation δ⁽ⁱ⁾ such that f(x₀⁽ⁱ⁾+δ⁽ⁱ⁾)>p can be obtained, by solving the following optimization problem,

$${δ}^{( i )} = {min}_{δ} {L}_{pred} ( {x}_{0}^{( i )} , δ ) + {L}_{dist} ( δ ) + {L}_{proto} ( {x}_{0}^{( i )} , δ ) , \qquad ( 4 )$$

$$such that$$

$${L}_{pred} ( {x}_{0}^{( i )} , δ ) = c max ( - f ( {x}_{0}^{( i )} , δ ) , - p ) , \qquad ( 5 )$$

$${L}_{dist} ( δ ) = β {‖ δ ‖}_{1} + {‖ δ ‖}_{2}^{2} ,$$

$${L}_{proto} ( {x}_{0}^{( i )} , δ ) = θ {‖ {x}_{0}^{( i )} + δ - proto ‖}_{2}^{2} , \qquad ( 6 )$$

$${δ}^{( i )} = {γ}^{( i )} {⊙}_{3} {x}_{0}^{( i )} ,$$

where proto is an instance of the training set classified as having T cells, defined by first building a k-d tree of training instances classified as having T cells and setting the k-nearest item in the tree (in terms of euclidean distance to x₀⁽ⁱ⁾) as proto. k=1 was used for all optimizations.

### Data Split

Code for model training, perturbation optimization and analysis are publicly available at github.com/neonine2/deepspace, the content of which is incorporated herein by reference in its entirety.

### Additional Information

### Assessment of T-Cell Masking Strategy

By masking T cells prior to model training, a new signal that is predictive of T cells may be inadvertently introduced, specifically that less cells in an image increases the probability of T cells being present. To assess the possibility of such an effect, the impact of random cell masking on the predicted probability of T-cell presence was studied. For each patch, three randomized versions were generated, where one of the cells was randomly selected to be masked for each version. Then, the difference in predicted probabilities and predicted label between the randomized patches and the original patch was computed (Table 4). Across all three data sets, no statistically significant difference in the predicted labels was seen, when comparing randomly-masked patches to original patches. A significant increase in the predicted probability value for CRC in the randomized images was found, although the mean change was very small at 1.38×10⁻⁴. These results suggested that the T-cell masking strategy did not introduce an artificial signal whereby simply removing cells at random would increase the chance that T cells were predicted to be present. In Table 4, p-value obtained from a one-sample T-test testing indicated whether the observed difference was significantly different from zero.

TABLE 4
DIFFERENCE IN PREDICTED PROBABILITY AND
PREDICTED POSITIVE LABELS BETWEEN RANDOMLY
MASKED PATCHES AND ORIGINAL PATCHES

| | Mean | | Mean | |
|---|---|---|---|---|
| Cancer type | difference | p-value | difference | p-value |
| Melanoma | −2.22 × 10⁻⁴ | 0.132 | −1.45 × 10⁻⁴ | 2.28 × 10⁻⁵ |
| Breast tumor | −1.93 × 10⁻⁵ | 0.739 | 6.96 × 10⁻⁶ | 0.588 |
| CRC | 8.95 × 10⁻⁵ | 0.433 | 1.38 × 10⁻⁴ | 2.48 × 10⁻¹³ |

### Choice of IMC Patch Size

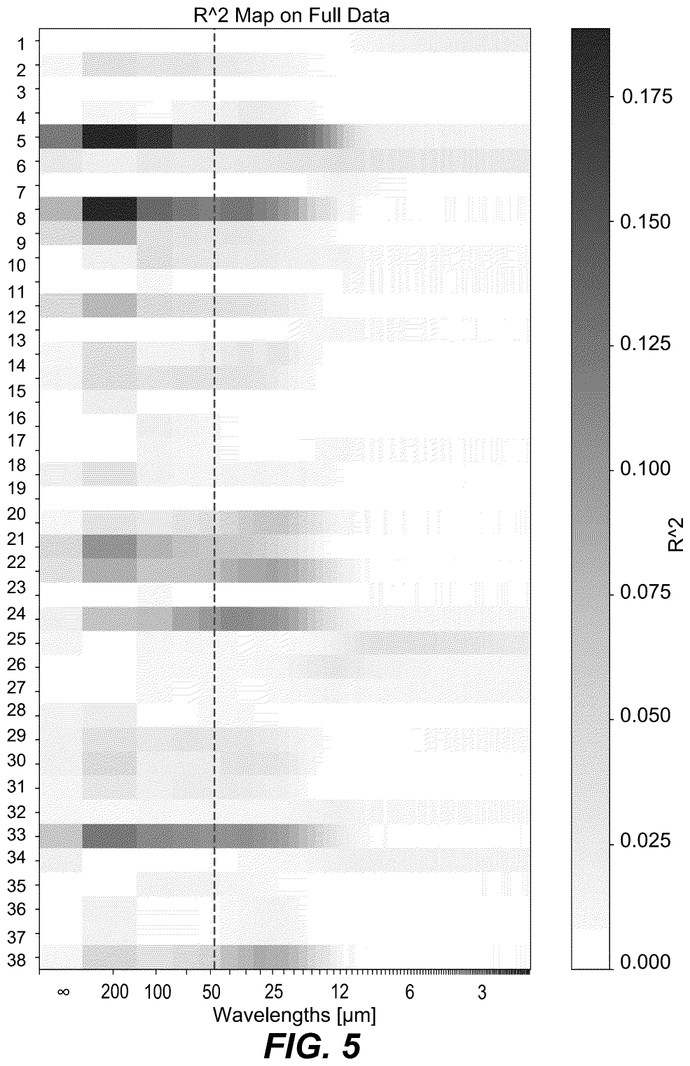

In order to obtain enough TME samples to train the classifier models, the inherent heterogeneity in the TME was taken advantage of and each tissue image was divided into 48 μm×48 μm patches. Each patch was treated as an independent sample during training. Spectral analysis was performed to study the relationship between spatial patterning of proteins at various length scales and CD8+ T-cell infiltration. Specifically, the power spectral density (PSD) of each tumor sample image was computed. The PSD showed the relative importance of patterning at various length scales (“wavelengths”) in the expression map. Then, the Pearson correlation between patterning of a protein at a given length scale and T-cell infiltration was computed. FIG. 5 shows there were significant information pertaining to T-cell infiltration at the selected patch size. For certain proteins such as HLA-DR and FSP1, significantly more information was present at longer length scales of around 200. This result suggested that for sufficient large sample size, the performance the classifier model disclosed herein can be improved by increasing the size of image patches.

TABLE 5
PERFORMANCE OF DIFFERENT CLASSIFIER MODELS TRAINED
ON MELANOMA, LIVER TUMOR, AND BREAST TUMOR IMC IMAGES
TO PREDICT THE PRESENCE OF T CELLS (p = 0.5)

| Cancer type | Model | Accuracy | Precision | Recall | F1 | AUROC | MCC |
|---|---|---|---|---|---|---|---|
| Melanoma | Linear | 0.84 | 0.83 | 0.37 | 0.51 | 0.67 | 0.48 |
| | MLP | 0.85 | 0.79 | 0.48 | 0.59 | 0.72 | 0.53 |
| | U-Net | 0.86 | 0.71 | 0.61 | 0.65 | 0.77 | 0.57 |
| Breast tumor | Linear | 0.82 | 0.57 | 0.16 | 0.24 | 0.57 | 0.23 |
| | MLP | 0.86 | 0.71 | 0.42 | 0.52 | 0.69 | 0.47 |
| | U-Net | 0.87 | 0.70 | 0.48 | 0.56 | 0.71 | 0.50 |
| CRC | Linear | 0.86 | 0.71 | 0.19 | 0.29 | 0.59 | 0.31 |
| | MLP | 0.88 | 0.67 | 0.50 | 0.57 | 0.73 | 0.51 |
| | U-Net | 0.90 | 0.68 | 0.65 | 0.66 | 0.80 | 0.60 |

TABLE 6
PERFORMANCE OF DIFFERENT CNN AND VIT MODELS
TRAINED ON IMC IMAGE PATCHES OF MELANOMA

| Model Name | U-Net | ResNet-18 | EfficientNet-B0 | MedViT |
|---|---|---|---|---|
| Number of | 12.8M | 11.2M | 4.1M | 31.3M |
| parameters | | | | |
| Test accuracy | 0.86 | 0.86 | 0.851 | 0.853 |

FIG. 5 depicts non-limiting exemplary correlation between each frequency band of each protein channel and T-cell infiltration level (proportion of CD8+ T-cell patches) across all IMC images for the breast cancer data set. The proteins are: 1, Histone H3; 2, SMA; 3, CK5; 4, CD38; 5, HLA-DR; 6, CK8-18; 7, CD15; 8, FSPI; 9, CD163; 10, ICOS; 11, OX40; 12, CD68; 13, HER2 (3B5); 14, CD3; 15, Podoplanin; 16, CD11c; 17, PD-1; 18, GITR; 19, CD16; 20, HERZ (D8F12); 21, CD45RA; 22, B2M; 23, CD45RO; 24, FOXP3; 25, CD20; 26, ER; 27, CD57; 28, Ki-67; 29, PDGFRB; 30, Caveolin-1; 31, CD4; 32, CD31-VWE; 33, CXCL12; 34, HLA-ABC; 35, panCK; 36, c-Caspase3; 37, DNA1; 38, DNA2; and 39, CD8. Dotted line indicates the patch size used.

### Example Counterfactual Optimization

Disclosed herein include methods for counterfactual optimization. In some embodiments, a method for counterfactual optimization is under control of a processor (e.g., a hardware processor or a virtual processor) and comprises: receiving spatial omics training data comprising a plurality of training images each comprising a plurality of molecule channels. The method can comprise: generating a training image label for each of the plurality of training images indicating presence of at least one T cell in the training image. The method can comprise: generating a plurality of masked training images from the plurality of training images with any T cell present in a training image of the plurality of training images masked in a masked training image of the plurality of masked training images generated. The method can comprise: training a model comprising a classifier with the plurality of masked training images as input and the training image label as output. The method can comprise: performing a counterfactual optimization to determine a tumor perturbation using a second plurality of images with no T cell present in each of the plurality of images. In some embodiments, the method comprises: receiving the second plurality of images with no T cell present in each of the plurality of images

In some embodiments, the spatial omics data is generated using imaging mass cytometry. In some embodiments, the spatial omics data comprises proteinomics data, transcriptomics data, or a combination thereof. In some embodiments, the spatial omics data comprises multi-omics training data.

In some embodiments, a training image of the plurality of training images is, is about, is at least, is at least about, is at most, or is at most about, 20, 25, 30, 35, 40, 45, 58, 50, 55, 60, 70, 80, 90, 100, 125, 150, 175, 200, 250, 300, or a number or a range between any two of these values, pixels in length (or width). In some embodiments, a training image of the plurality of training images corresponds to a section (or a region) that is, is about, is at least, is at least about, is at most, or is at most about, 20 μm, 25 μm, 30 μm, 35 μm, 40 μm, 45 μm, 58 μm, 50 μm, 55 μm, 60 μm, 70 μm, 80 μm, 90 μm, 100 μm, 125 μm, 150 μm, 175 μm, 200 μm, 250 μm, 300 μm, or a number or a range between any two of these values, in length (or width).

In some embodiments, the plurality of molecule channels comprises, comprises about, comprises at least, comprises at least about, comprises at most, or comprises at most about, 10, 15, 20, 25, 30, 35, 37 40, 41, 45, 50, 55, 60, 65, 70, 75, 80, 85, 90, 95, 100, 125, 150, 175, 200, 250, 300, 400, 500, or a number or a range between any two of these values, channels. In some embodiments, each of the plurality of molecule channels corresponds to a different protein. In some embodiments, the molecules comprise chemokines. In some embodiments, the molecules comprise CXCL9, CXCL10, CCL18, CCL22, or a combination thereof. In some embodiments, the molecules comprise Histone H3, SMA, CK5, CD38, HLA-DR, CK8-18, CD15, FSPI, CD163, ICOS, OX40, CD68, HER2 (3B5), CD3, Podoplanin, CD11c, PD-1, GITR, CD16, HERZ (D8F12), CD45RA, B2M, CD45RO, FOXP3, CD20, ER, CD57, Ki-67, PDGFRB, Caveolin-1, CD4, CD31-VWE, CXCL12, HLA-ABC, panCK, c-Caspase3, DNA1, DNA2, CD8, or a combination thereof.

In some embodiments, the training image label is a binary value. The training image label being 0 can indicate absence of any T cell in a corresponding training image of the training image label. The training image label being 1 can indicate presence of at least one T cell in a corresponding training image of the training image label. In some embodiments, generating the training image label comprises: generating the training image label by clustering cells in the training image and one or more other (e.g., 2, 3, 4, 5, 10, 25, 50, 75, 100, or all or substantially all) training images of the plurality of training images.

In some embodiments, masked pixels in one, one or more, or each of the plurality of masked training images comprise values of 0. Masked pixels in one, one or more, or each of the plurality of masked training images can comprise an average value of pixels (e.g., some pixels, pixels excluding the masked pixels, or all pixels) in the masked training image or the corresponding training image. In some embodiments, the plurality of masked training images comprises, for each of the plurality training images, the training image if no T cell is present in the training image, or the training image with any T cell present in the training image masked.

In some embodiments, the model comprises a fully-connected layer connected to a last layer of the classifier. The fully-connected layer can output a value between 0 and 1. The model can output a value of 0 if the output of the fully-connected layer is below a threshold value. The model can output a value of 1 if the output of the fully-connected layer is at least the threshold value. In some embodiments, wherein the threshold value is 0.9 (or is, is about, is at least, is at least about, is at most, or is at most about, 7, 7.5, 8, 8.5, 8.6, 8.7, 8.8, 8.9, 9, 9.1, 9.2, 9.3, 9.4, 9.5, or a number or a range between any two of these values). In some embodiments, the method comprises: determining the threshold value, optionally wherein determining the threshold value using root mean squared error (RMSE).

In some embodiments, the classifier comprises a neural network, a deep neural network, a convolutional neural network, a fully convolutional neural network, or a combination thereof In some embodiments, the classifier comprises U-Net, Reset-18, EfficientNet-B0, MedViT, or a combination thereof. In some embodiments, training the model comprises training the model using stochastic gradient decent and/or T cell prediction loss. In some embodiments, the spatial omics training data comprises the second plurality of images with no T cell present in each the image. In some embodiments, the plurality of training images comprises the second plurality of images with no T cell present in the image. In some embodiments, the second plurality of images with no T cell present in the image comprises no training image of the plurality of training images.

In some embodiments, performing the counterfactual optimization comprises: performing the counterfactual optimization using a term corresponding to increasing predicted probability of T cells, a term corresponding to minimizing change, and/or a term corresponding to a shift closer to training data. In some embodiments, a tumor perturbation comprises, for each of the second plurality of images, a change in an intensity in one or more (e.g., 1, 2, 3, 4, 5, 6, 7, 8, 9, 10, 15, 20, 25, 30, 35, 40, 45, 50, 60, 70, 80, 90, or 100, or all, or a number or a range between any two of these values) of the plurality of molecule channels. In some embodiments, method comprises: applying the tumor perturbation to a plurality of test images with a T-cell distribution to determine a perturbed T-cell distribution. In some embodiments, the method comprises: testing the tumor perturbation, for example, on cells, an organoid, a tissue, or a combination thereof.

### Neural Network

A neural network (NN), such as a convolutional neural network (CNN) or a deep neural network (DNN), can be used for any method herein, such as counterfactual optimization.

A layer of a neural network (NN), such as a deep neural network (DNN), can apply a linear or non-linear transformation to its input to generate its output. A neural network layer can be a normalization layer, a convolutional layer, a softsign layer, a rectified linear layer, a concatenation layer, a pooling layer, a recurrent layer, an inception-like layer, or any combination thereof. The normalization layer can normalize the brightness of its input to generate its output with, for example, L2 normalization. The normalization layer can, for example, normalize the brightness of a plurality of images with respect to one another at once to generate a plurality of normalized images as its output. Non-limiting examples of methods for normalizing brightness include local contrast normalization (LCN) or local response normalization (LRN). Local contrast normalization can normalize the contrast of an image non-linearly by normalizing local regions of the image on a per pixel basis to have a mean of zero and a variance of one (or other values of mean and variance). Local response normalization can normalize an image over local input regions to have a mean of zero and a variance of one (or other values of mean and variance). The normalization layer may speed up the training process.

A convolutional neural network (CNN) can be a NN with one or more convolutional layers, such as, 5, 6, 7, 8, 9, 10, or more. The convolutional layer can apply a set of kernels that convolve its input to generate its output. The softsign layer can apply a softsign function to its input. The softsign function (softsign(x)) can be, for example, (x/(1+|x|)). The softsign layer may neglect impact of per-element outliers. The rectified linear layer can be a rectified linear layer unit (ReLU) or a parameterized rectified linear layer unit (PReLU). The ReLU layer can apply a ReLU function to its input to generate its output. The ReLU function ReLU(x) can be, for example, max(0, x). The PReLU layer can apply a PReLU function to its input to generate its output. The PReLU function PReLU(x) can be, for example, x if x≥0 and ax if x<0, where a is a positive number. The concatenation layer can concatenate its input to generate its output. For example, the concatenation layer can concatenate four 5×5 images to generate one 20×20 image. The pooling layer can apply a pooling function which down samples its input to generate its output. For example, the pooling layer can down sample a 20×20 image into a 10×10 image. Non-limiting examples of the pooling function include maximum pooling, average pooling, or minimum pooling.

At a time point t, the recurrent layer can compute a hidden state s(t), and a recurrent connection can provide the hidden state s(t) at time t to the recurrent layer as an input at a subsequent time point t+1. The recurrent layer can compute its output at time t+1 based on the hidden state s(t) at time t. For example, the recurrent layer can apply the softsign function to the hidden state s(t) at time t to compute its output at time t+1. The hidden state of the recurrent layer at time t+1 has as its input the hidden state s(t) of the recurrent layer at time t. The recurrent layer can compute the hidden state s(t+1) by applying, for example, a ReLU function to its input. The inception-like layer can include one or more of the normalization layer, the convolutional layer, the softsign layer, the rectified linear layer such as the ReLU layer and the PReLU layer, the concatenation layer, the pooling layer, or any combination thereof.

The number of layers in the NN can be different in different implementations. For example, the number of layers in a NN can be 10, 20, 30, 40, or more. For example, the number of layers in the DNN can be 50, 100, 200, or more. The input type of a deep neural network layer can be different in different implementations. For example, a layer can receive the outputs of a number of layers as its input. The input of a layer can include the outputs of five layers. As another example, the input of a layer can include 1% of the layers of the NN. The output of a layer can be the inputs of a number of layers. For example, the output of a layer can be used as the inputs of five layers. As another example, the output of a layer can be used as the inputs of 1% of the layers of the NN.

The input size or the output size of a layer can be quite large. The input size or the output size of a layer can be n×m, where n denotes the width and m denotes the height of the input or the output. For example, n or m can be 11, 21, 31, or more. The channel sizes of the input or the output of a layer can be different in different implementations. For example, the channel size of the input or the output of a layer can be 4, 16, 32, 64, 128, or more. The kernel size of a layer can be different in different implementations. For example, the kernel size can be n×m, where n denotes the width and m denotes the height of the kernel. For example, n or m can be 5, 7, 9, or more. The stride size of a layer can be different in different implementations. For example, the stride size of a deep neural network layer can be 3, 5, 7 or more.

In some embodiments, a NN can refer to a plurality of NNs that together compute an output of the NN. Different NNs of the plurality of NNs can be trained for different tasks. Outputs of NNs of the plurality of NNs can be computed to determine an output of the NN. For example, an output of a NN of the plurality of NNs can include a likelihood score. The output of the NN including the plurality of NNs can be determined based on the likelihood scores of the outputs of different NNs of the plurality of NNs.

### Execution Environment

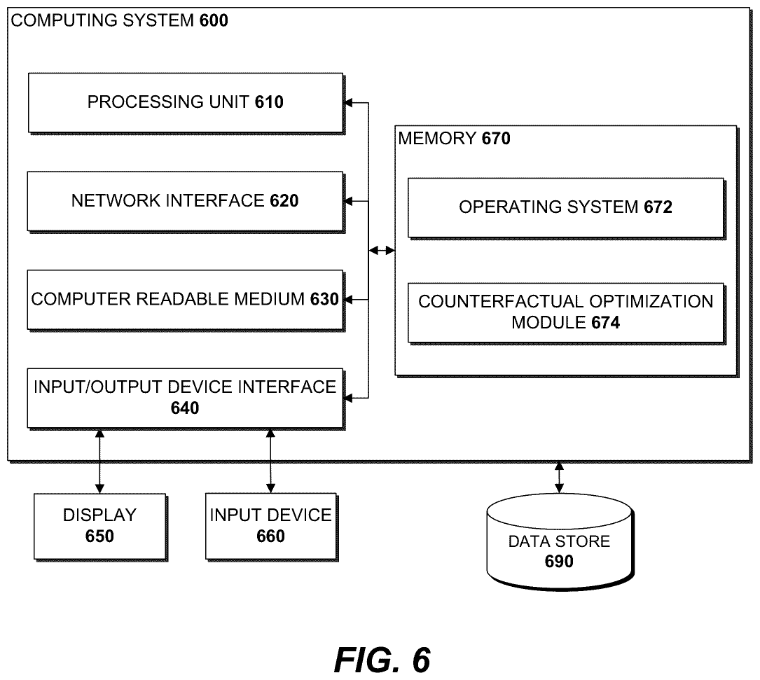

FIG. 6 depicts a general architecture of an example computing device 600 that can be used in some embodiments to execute the processes and implement the features described herein. The general architecture of the computing device 600 depicted in FIG. 6 includes an arrangement of computer hardware and software components. The computing device 600 may include many more (or fewer) elements than those shown in FIG. 6. It is not necessary, however, that all of these generally conventional elements be shown in order to provide an enabling disclosure. As illustrated, the computing device 600 includes a processing unit 610, a network interface 620, a computer readable medium drive 630, an input/output device interface 640, a display 650, and an input device 660, all of which may communicate with one another by way of a communication bus. The network interface 620 may provide connectivity to one or more networks or computing systems. The processing unit 610 may thus receive information and instructions from other computing systems or services via a network. The processing unit 610 may also communicate to and from memory 670 and further provide output information for an optional display 650 via the input/output device interface 640. The input/output device interface 640 may also accept input from the optional input device 660, such as a keyboard, mouse, digital pen, microphone, touch screen, gesture recognition system, voice recognition system, gamepad, accelerometer, gyroscope, or other input device.

The memory 670 may contain computer program instructions (grouped as modules or components in some embodiments) that the processing unit 610 executes in order to implement one or more embodiments. The memory 670 generally includes RAM, ROM and/or other persistent, auxiliary or non-transitory computer-readable media. The memory 670 may store an operating system 672 that provides computer program instructions for use by the processing unit 610 in the general administration and operation of the computing device 600. The memory 670 may further include computer program instructions and other information for implementing aspects of the present disclosure.

For example, in one embodiment, the memory 670 includes a counterfactual optimization module 674 for training a classifier (or a model comprising a classifier) for counterfactual optimization and/or for performing counterfactual optimization. In addition, memory 670 may include or communicate with the data store 690 and/or one or more other data stores that store, for example, input, results (e.g., intermediate results or final results), and outputs of classifier training and counterfactual optimization, such as spatial omics data, images (e.g., training images, test images), image labels, a classifier (or a model comprising a classifier) or a portion thereof, perturbations (e.g., perturbations for images and tumor perturbations), perturbed images, and/or perturbed T-cell distribution.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A system for counterfactual optimization comprising:
non-transitory memory configured to store:
executable instructions; and
spatial omics training data comprising a plurality of training images each comprising a plurality of molecule channels; and
a hardware processor in communication with the non-transitory memory, the hardware processor programmed by the executable instructions to perform:
generating a training image label for each of the plurality of training images indicating presence of at least one T cell in the training image;
generating a plurality of masked training images from the plurality of training images with any T cell present in a training image of the plurality of training images masked in a masked training image of the plurality of masked training images generated;
training a model comprising a classifier with the plurality of masked training images as input and the training image label as output; and
performing a counterfactual optimization to determine a tumor perturbation using a second plurality of images with no T cell present in each of the plurality of images.

2. The system of claim 1, wherein the T cell is CD8+ T cell.

3. The system of claim 1, wherein the spatial omics training data comprises spatial omics data generated from a tumor sample of a subject and/or a tumor sample for each of a plurality of subjects or from a plurality of tumor samples of a subject or a plurality of tumor samples for each of a plurality of subjects.

4. (canceled)

5. The system of claim 3, wherein a subject comprises a mammal, or a human.

6. The system of claim 5, wherein the subject is a cancer subject.

7. The system of claim 1, wherein the spatial omics data is generated using imaging mass cytometry.

8. The system of claim 1, wherein the spatial omics data comprises proteomics data, transcriptomics data, or a combination thereof.

9. The system of claim 1, wherein a training image of the plurality of training images is 48 pixels by 48 pixels in size, and/or a training image of the plurality of training images corresponds to a section of 48 μm by 48 μm in size.

10. (canceled)

11. (canceled)

12. The system of claim 1, wherein each of the plurality of molecule channels corresponds to a different protein.

13. The system of claim 1, wherein the training image label is a binary value, wherein 0 indicates absence of any T cell in a corresponding training image of the training image label, and/or wherein 1 indicates presence of at least one T cell in a corresponding training image of the training image label.

14. The system of claim 1, wherein generating the training image label comprises: generating the training image label by clustering cells in the training image and one or more other training images of the plurality of training images.

15. The system of claim 1, wherein masked pixels in each of the plurality of masked training images comprise values of 0 or an average value of pixels in the masked training image or the corresponding training image, or wherein the plurality of masked training images comprises, for each of the plurality training images, the training image if no T cell is present in the training image, or the training image with any T cell present in the training image masked.

16. (canceled)

17. The system of claim 1, wherein the model comprises a fully-connected layer connected to a last layer of the classifier, wherein the fully-connected layer outputs a value between 0 and 1, and/or wherein the model outputs a value of 0 if the output of the fully-connected layer is below a threshold value and a value of 1 if the output of the fully-connected layer is at least the threshold value.

18. (canceled)

19. The system of claim 1, wherein the hardware processor is programmed by the executable instructions to perform: (i) determining the threshold value, optionally wherein determining the threshold value using root mean squared error (RMSE), and/or (ii) applying the tumor perturbation to a plurality of test images with a T-cell distribution to determine a perturbed T-cell distribution.

20. The system of claim 1, wherein the classifier comprises a neural network, a deep neural network, a convolutional neural network, a fully convolutional neural network, or a combination thereof.

21. The system of claim 1, wherein the classifier comprises U-Net, Reset-18, EfficientNet-B0, MedViT, or a combination thereof.

22. The system of claim 1, wherein training the model comprises training the model using stochastic gradient decent and/or T cell prediction loss.

23. The system of claim 1, wherein (i) the spatial omics training data comprises the second plurality of images with no T cell present in each the image, (ii) the plurality of training images comprises the second plurality of images with no T cell present in the image; and/or (iii) the second plurality of images with no T cell present in the image comprises no training image of the plurality of training images.

24. (canceled)

25. (canceled)

26. The system of claim 1, wherein the counterfactual optimization comprises a term corresponding to increasing predicted probability of T cells, a term corresponding to minimizing change, and/or a term corresponding to a shift closer to training data.

27. The system of claim 1, wherein a tumor perturbation comprises, for each of the second plurality of images, a change in an intensity in each of one or more of the plurality of molecule channels.

28.-58. (canceled)
